(19) United States
(12) Patent Application Publication
(10) Pub. No.: US 2022/0330597 A1
(43) Pub. Date: Oct. 20, 2022

(54) COMPOSITIONS AND METHODS FOR EXTENDING LIFESPAN

(71) Applicant: MarvelBiome, Inc., Woburn, MA (US)

(72) Inventors: Jothi Amaranath Govindan, Malden, MA (US); Elamparithi Jayamani, Melrose, MA (US); Priti H. Chatter, Concord, MA (US)

(21) Appl. No.: 17/765,820

(22) PCT Filed: Sep. 23, 2020
(86) PCT No.: PCT/US20/52251

§ 371 Date: Mar. 31, 2022

Related U.S. Application Data
(60) Provisional application No. 62/909,186, filed on Oct. 1, 2019.

(51) Int. Cl. A23L 33/135; A61P 21/00; A61P 43/00; C12N 1/20; A61K 35/74

(52) U.S. Cl. CPC A23L 33/135; A61P 21/00; A61P 43/00; C12N 1/20; A61K 35/74

(57) ABSTRACT

Compositions are provided that extend the lifespan of a subject, and/or reduce or delay the onset of at least one age-associated symptom or condition. In some embodiments, compositions comprise: at least one bacterial strain or extract(s) or component(s) thereof and an excipient. In some embodiments, at least one bacterial strain comprises Gluconobacter spp., Acetobacter spp., Gluconoacaetobacter spp., Acidomonas spp., Ameyamaea spp., Asaia spp., Granulibacter spp., Kozakia spp., Neoasaia spp., Neokomagataea spp., Saccharibacter spp., Swaminathania spp., Tanticharoenia spp., or a combination thereof. Methods of making and using compositions disclosed herein are also provided.

### CROSS REFERENCE TO RELATED APPLICATIONS

The present application claims priority to U.S. Provisional Patent Application No. 62/909,186, filed Oct. 1, 2019, the entire contents of which is hereby incorporated by reference in its entirety.

### BACKGROUND

Aging is a complex process that affects every cellular processes and lead to a wide variety of altered functions.

### SUMMARY

The present disclosure provides compositions comprising at least one bacterial strain or extract(s) or component(s) thereof, and an excipient.

In some embodiments, at least one bacterial strain comprises a Gluconobacter spp., Acetobacter spp., Gluconoacaetobacter spp., Acidomonas spp., Ameyamaea spp., Asaia spp., Granulibacter spp., Kozakia spp., Neoasaia spp., Neokomagataea spp., Saccharibacter spp., Swaminathania spp., Tanticharoenia spp., or a combination thereof In some embodiments, at least one bacterial strain comprises Gluconobacter albidus, Gluconobacter cerinus, Gluconobacter frateruii, Gluconobacter japonicus, Gluconobacter kondonii, Gluconobacter nephelii, Gluconobacter oxydans, Gluconoacetobacter diazotrophicus, Gluconoacetobacter hansenii, Gluconoacetobacter saccharivorans, Acetobacter aceti, Acetobacter malorum, or a combination thereof. In some embodiments, at least one bacterial strain comprises Gluconacetobacter hansenii, Gluconobacter oxydans, Acetobacter aceti, or a combination thereof. In some embodiments, at least one bacterial strain comprises Gluconacetobacter hansenii.

In some embodiments, an excipient is or comprises an inactive (e.g., non-biologically active) agent. An excipient may be included in a composition, for example, to provide or contribute to a desired consistency or stabilizing effect. In some embodiments, excipients may include, for example, starch, glucose, lactose, sucrose, gelatin, malt, rice, flour, chalk, silica gel, sodium stearate, glycerol monostearate, talc, sodium chloride, dried skim milk, glycerol, propylene, glycol, water, or ethanol.

In some embodiments, a composition is formulated for oral administration. In some embodiments, a composition is a food, a beverage, a feed composition, or a nutritional supplement. In some embodiments, a composition is a liquid, syrup, tablet, troche, gummy, capsule, powder, gel, or film. In some embodiments, a composition is a pharmaceutical composition. In some embodiments, a composition is an enteric-coated formulation.

In some embodiments, at least one bacterial strain or extract(s) or component(s) thereof is characterized in that, when administered to a C. elegans culture comprising C. elegans animals, an average life span of the C. elegans animals in the C. elegans culture is extended by at least 20%, at least 25%, at least 30%, at least 35%, at least 40%, at least 45%, or at least 50%, as compared to that of C. elegans animals in a comparable C. elegans culture without administration of the at least one bacterial strain or extract or component thereof.

In some embodiments, at least one bacterial strain or extract(s) or component(s) thereof is characterized in that, when administered to a C. elegans culture comprising C. elegans animals, average pharyngeal pumping activity of the C. elegans animals in the C. elegans culture is increased by at least 20%, at least 25%, at least 30%, at least 35%, at least 40%, at least 45%, or at least 50%, as compared to that of C. elegans animals in a comparable C. elegans culture without administration of the at least one bacterial strain or extract(s) or component(s) thereof.

In some embodiments, at least one bacterial strain or extract(s) or component(s) thereof is characterized in that, when administered to a C. elegans culture comprising C. elegans animals, average locomotion rate of the C. elegans animals in the C. elegans culture is increased by at least 20%, at least 25%, at least 30%, at least 35%, at least 40%, at least 45%, or at least 50%, as compared to that of C. elegans animals in a comparable C. elegans culture without administration of the at least one bacterial strain or extract(s) or component(s) thereof.

In some embodiments, at least one bacterial strain or extract(s) or component(s) thereof is characterized in that, when administered to a C. elegans culture comprising C. elegans animals, fertility of the C. elegans animals in the C. elegans culture is decreased by at least 20%, at least 25%, at least 30%, at least 35%, at least 40%, at least 45%, or at least 50%, as compared to that of C. elegans animals in a comparable C. elegans culture without administration of the at least one bacterial strain or extract(s) or component(s) thereof.

In some embodiments, at least one bacterial strain or extract(s) or component(s) thereof is characterized in that, when a C. elegans culture comprising C. elegans animals is exposed to Ultra Violet irradiation, average survival time of the C. elegans animals in the C. elegans culture to which the at least one bacterial strain or extract(s) or component(s) thereof has been administered is increased by at least 20%, at least 25%, at least 30%, at least 35%, at least 40%, at least 45%, or at least 50%, as compared to that of C. elegans animals in a comparable C. elegans culture without administration of the at least one bacterial strain or extract(s) or component(s) thereof.

In some embodiments, at least one bacterial strain or extract(s) or component(s) thereof is characterized in that, when a C. elegans culture comprising C. elegans animals is exposed to an elevated temperature, average survival time of C. elegans animals in the C. elegans culture to which the at least one bacterial strain or extract(s) or component(s) thereof has been administered is increased by at least 20%, at least 25%, at least 30%, at least 35%, at least 40%, at least 45%, or at least 50%, as compared to that C. elegans animals in of a comparable C. elegans culture without administration of the at least one bacterial strain or extract(s) or component(s) thereof. In some embodiments, an elevated temperature is at least 37° C., at least 40° C., at least 45° C., at least 50° C., at least 55° C., at least 60° C., at least 65° C., at least 70° C., at least 75° C., or at least 80° C. In some embodiments, an elevated temperature is 50° C.-65° C., 65° C.-80° C., or 80° C.-120° C.

In some embodiments, at least one bacterial strain or extract(s) or component(s) thereof is characterized in that, when administered to a C. elegans culture comprising C. elegans animals, average amount of intestinal fat observed in the C. elegans animals \is decreased by at least 20%, at least 25%, at least 30%, at least 35%, at least 40%, at least 45%, or at least 50%, as compared to that of C. elegans animals in a comparable C. elegans culture without administration of the at least one bacterial strain or extract(s) or component(s) thereof.

In some embodiments, C. elegans animals are adult C. elegans animals. In some embodiments, C. elegans animals are at least 5 days old.

The present disclosure provides methods comprising administering a composition described herein to a subject a composition.

In some embodiments, a method is a method of extending lifespan of a subject. In some embodiments, the life span of a subject is extended by at least 20%, at least 25%, at least 30%, at least 35%, at least 40%, at least 45%, or at least 50%, as compared to that of a comparable subject without administration of the composition.

In some embodiments, a method is a method of reducing or delaying the onset of at least one age-associated symptom or condition in a subject. In some embodiments, at least one age-associated symptom or condition is reduced or delayed in a subject by at least 20%, at least 25%, at least 30%, at least 35%, at least 40%, at least 45%, or at least 50%, as compared to that of a comparable subject without administration of the composition. In some embodiments, at least one age-associated symptom or condition is or comprises a decline in muscle and/or neuromuscular function of a subject. In some embodiments, at least one age-associated symptom or condition is or comprises dysregulation of lipid metabolism.

In some embodiments, a subject is at least 30 years old, at least 35 years old, at least 40 years old, at least 45 years old, at least 50 years old, at least 55 years old, at least 60 years old, at least 65 years old, at least 70 years old, or at least at least 75 years old. In some embodiments, a subject is an elderly subject.

In some embodiments, a subject is a mammal. In some embodiments, a mammal is a non-human primate (e.g, a higher primate), a sheep, a dog, a rodent (e.g., a mouse or rat), a guinea pig, a goat, a pig, a cat, a rabbit, or a cow. In some embodiments, a mammal is a human.

In some embodiments, a method comprises administering comprises administering a sufficient amount of the microbes to colonize the subject's microbiome.

In some embodiments, the step of administering comprises ingesting.

The present disclosure provides uses of compositions disclosed herein for extending the life span of a subject. The present disclosure provides uses of at least one bacterial strain or extract(s) or component(s) thereof for extending the life span of a subject. In some embodiments, at least one bacterial strain comprises Gluconobacter spp., Acetobacter spp., Gluconoacaetobacter spp., Acidomonas spp., Ameyamaea spp., Asaia spp., Granulibacter spp., Kozakia spp., Neoasaia spp., Neokomagataea spp., Saccharibacter spp., Swaminathania spp., Tanticharoenia spp., or a combination thereof. In some embodiments, at least one bacterial strain comprises Gluconacetobacter hansenii, Gluconobacter oxydans, Acetobacter aceti, or a combination thereof. In some embodiments, at least one bacterial strain comprises Gluconacetobacter hansenii.

The present disclosure provides uses of compositions described herein for reducing or delaying the onset of at least one age-associated symptom or condition in a subject. The present disclosure provides uses of at least one bacterial strain or extract(s) or component(s) thereof for reducing or delaying the onset of at least one age-associated symptom or condition in a subject. In some embodiments, at least one bacterial strain comprises Gluconobacter spp., Acetobacter spp., Gluconoacaetobacter spp., Acidomonas spp., Ameyamaea spp Asaia spp., Granulibacter spp., Kozakia spp., Neoasaia spp., Neokomagataea spp., Saccharibacter spp., Swaminathania spp., Tanticharoenia spp., or a combination thereof. In some embodiments, at least one bacterial strain comprises Gluconacetobacter hansenii, Gluconobacter oxydans, Acetobacter aceti, or a combination thereof. In some embodiments, at least one bacterial strain comprises Gluconacetobacter hansenii.

In some embodiments, at least one age-associated symptom or condition is reduced or delayed in the subject by at least 20%, at least 25%, at least 30%, at least 35%, at least 40%, at least 45%, or at least 50%, as compared to that of a comparable subject without administration of the composition.

In some embodiments, at least one age-associated symptom or condition is or comprises a decline in muscle and/or neuromuscular function of the subject. In some embodiments, at least one age-associated symptom or condition is or comprises dysregulation of lipid metabolism.

In some embodiments, a subject is at least 30 years old, at least 35 years old, at least 40 years old, at least 45 years old, at least 50 years old, at least 55 years old, at least 60 years old, at least 65 years old, at least 70 years old, or at least at least 75 years old. In some embodiments, a subject is an elderly subject.

In some embodiments, a subject is a mammal. In some embodiments, a mammal is a non-human primate (e.g, a higher primate), a sheep, a dog, a rodent (e.g., a mouse or rat), a guinea pig, a goat, a pig, a cat, a rabbit, or a cow. In some embodiments, a mammal is a human.

The present disclosure provides uses of compositions described herein for treating a subject who has or is at risk of developing a disease or disorder associated with premature aging. The present disclosure provides uses of at least one bacterial strain or extract(s) or component(s) thereof for treating a subject who has or is at risk of developing a disease or disorder associated with premature aging. In some embodiments, a disease or disorder is Bloom syndrome, Bockayne Syndrome, Hutchinson-Gilford progeria syndrome, mandibuloacral dysplasia with type A lipodystrophy, progeria, progeroid syndrome, Rothmund-Thomson syndrome, Seip syndrome, or Werner syndrome.

The present disclosure provides methods of characterizing the ability of one or more microbial strains to modify the life span of a subject, an age-associated symptom, and/or an age-associated condition, comprising (a) adding a plurality of microbial strains of a mammalian microbiome to a plurality of C. elegans cultures, wherein a different microbial strain is added to each C. elegans culture, and wherein each culture comprises C. elegans animals of the same C. elegans strain, and (b) determining whether each microbial strain of the plurality affects one or more parameters of the C. elegans animals of each culture, wherein the one or more parameters are associated with aging, an age-associated symptom, and/or an age-associated condition.

The present disclosure provides uses of C. elegans animals for characterizing the ability of one or more one or more microbial strains to modify the life span of a subject, an age-associated symptom, and/or an age-associated condition.

The present disclosure provides methods of making a composition as described herein comprising combining at least one bacterial strain or extract(s) or component(s) thereof, and the excipient.

### DEFINITIONS

The scope of the present invention is defined by the claims appended hereto and is not limited by certain embodiments described herein. Those skilled in the art, reading the present specification, will be aware of various modifications that may be equivalent to such described embodiments, or otherwise within the scope of the claims. In general, terms used herein are in accordance with their understood meaning in the art, unless clearly indicated otherwise. Explicit definitions of certain terms are provided below; meanings of these and other terms in particular instances throughout this specification will be clear to those skilled in the art from context.

Use of ordinal terms such as “first,” “second,” “third,” etc., in the claims to modify a claim element does not by itself connote any priority, precedence, or order of one claim element over another or the temporal order in which acts of a method are performed, but are used merely as labels to distinguish one claim element having a certain name from another element having a same name (but for use of the ordinal term) to distinguish the claim elements.

The articles “a” and “an,” as used herein, should be understood to include the plural referents unless clearly indicated to the contrary. Claims or descriptions that include “or” between one or more members of a group are considered satisfied if one, more than one, or all of the group members are present in, employed in, or otherwise relevant to a given product or process unless indicated to the contrary or otherwise evident from the context. In some embodiments, exactly one member of a group is present in, employed in, or otherwise relevant to a given product or process. In some embodiments, more than one, or all group members are present in, employed in, or otherwise relevant to a given product or process. It is to be understood that the invention encompasses all variations, combinations, and permutations in which one or more limitations, elements, clauses, descriptive terms, etc., from one or more of the listed claims is introduced into another claim dependent on the same base claim (or, as relevant, any other claim) unless otherwise indicated or unless it would be evident to one of ordinary skill in the art that a contradiction or inconsistency would arise. Where elements are presented as lists (e.g., in Markush group or similar format), it is to be understood that each subgroup of the elements is also disclosed, and any element(s) can be removed from the group. It should be understood that, in general, where embodiments or aspects are referred to as “comprising” particular elements, features, etc., certain embodiments or aspects “consist,” or “consist essentially of,” such elements, features, etc. For purposes of simplicity, those embodiments have not in every case been specifically set forth in so many words herein. It should also be understood that any embodiment or aspect can be explicitly excluded from the claims, regardless of whether the specific exclusion is recited in the specification.

Administration: As used herein, the term “administration” typically refers to the administration of a composition to a subject or system to achieve delivery of an agent to the subject or system. In some embodiments, the agent is, or is included in, the composition; in some embodiments, the agent is generated through metabolism of the composition or one or more components thereof. Those of ordinary skill in the art will be aware of a variety of routes that may, in appropriate circumstances, be utilized for administration to a subject, for example a human. For example, in some embodiments, administration may be ocular, oral, parenteral, topical, etc. In some particular embodiments, administration may be bronchial (e.g., by bronchial instillation), buccal, dermal (which may be or comprise, for example, one or more of topical to the dermis, intradermal, interdermal, transdermal, etc), enteral, intra-arterial, intradermal, intragastric, intramedullary, intramuscular, intranasal, intraperitoneal, intrathecal, intravenous, intraventricular, within a specific organ (e. g. intrahepatic), mucosal, nasal, oral, rectal, subcutaneous, sublingual, topical, tracheal (e.g., by intratracheal instillation), vaginal, vitreal, etc. In many embodiments provided by the present disclosure, administration is oral administration. In some embodiments, administration may involve only a single dose. In some embodiments, administration may involve application of a fixed number of doses. In some embodiments, administration may involve dosing that is intermittent (e.g., a plurality of doses separated in time) and/or periodic (e.g., individual doses separated by a common period of time) dosing. In some embodiments, administration may involve continuous dosing (e.g., perfusion) for at least a selected period of time. Administration of cells can be by any appropriate route that results in delivery to a desired location in a subject where at least a portion of the delivered cells or components of the cells remain viable. A period of viability of cells after administration to a subject can be as short as a few hours, e.g., twenty-four hours, to a few days, to as long as several years, i.e., long-term engraftment. In some embodiments, administration comprises delivery of a bacterial extract or preparation comprising one or more bacterial metabolites and/or byproducts but lacking fully viable bacterial cells.

Analog: As used herein, the term “analog” refers to a substance that shares one or more particular structural features, elements, components, or moieties with a reference substance. Typically, an “analog” shows significant structural similarity with the reference substance, for example sharing a core or consensus structure, but also differs in certain discrete ways. In some embodiments, an analog is a substance that can be generated from the reference substance, e.g., by chemical manipulation of the reference substance. In some embodiments, an analog is a substance that can be generated through performance of a synthetic process substantially similar to (e.g., sharing a plurality of steps with) one that generates the reference substance. In some embodiments, an analog is or can be generated through performance of a synthetic process different from that used to generate the reference substance.

Approximately: As applied to one or more values of interest, includes to a value that is similar to a stated reference value. In certain embodiments, the term “approximately” or “about” refers to a range of values that fall within ±10% (greater than or less than) of the stated reference value unless otherwise stated or otherwise evident from the context (except where such number would exceed 100% of a possible value).

Comparable: As used herein, the term “comparable” refers to two or more agents, entities, situations, sets of conditions, subjects, etc., that may not be identical to one another but that are sufficiently similar to permit comparison therebetween so that one skilled in the art will appreciate that conclusions may reasonably be drawn based on differences or similarities observed. In some embodiments, comparable sets of conditions, circumstances, individuals, or populations are characterized by a plurality of substantially identical features and one or a small number of varied features. Those of ordinary skill in the art will understand, in context, what degree of identity is required in any given circumstance for two or more such agents, entities, situations, sets of conditions, etc. to be considered comparable. For example, those of ordinary skill in the art will appreciate that sets of circumstances, individuals, or populations are comparable to one another when characterized by a sufficient number and type of substantially identical features to warrant a reasonable conclusion that differences in results obtained or phenomena observed under or with different sets of circumstances, individuals, or populations are caused by or indicative of the variation in those features that are varied.

Conservative: As used herein, refers to instances when describing a conservative amino acid substitution, including a substitution of an amino acid residue by another amino acid residue having a side chain R group with similar chemical properties (e.g., charge or hydrophobicity). In general, a conservative amino acid substitution will not substantially change the functional properties of interest of a protein, for example, the ability of a receptor to bind to a ligand. Examples of groups of amino acids that have side chains with similar chemical properties include: aliphatic side chains such as glycine (Gly, G), alanine (Ala, A), valine (Val, V), leucine (Leu, L), and isoleucine (Ile, I); aliphatic-hydroxyl side chains such as serine (Ser, S) and threonine (Thr, T); amide-containing side chains such as asparagine (Asn, N) and glutamine (Gln, Q); aromatic side chains such as phenylalanine (Phe, F), tyrosine (Tyr, Y), and tryptophan (Trp, W); basic side chains such as lysine (Lys, K), arginine (Arg, R), and histidine (His, H); acidic side chains such as aspartic acid (Asp, D) and glutamic acid (Glu, E); and sulfur-containing side chains such as cysteine (Cys, C) and methionine (Met, M). Conservative amino acids substitution groups include, for example, valine/leucine/isoleucine (Val/Leu/Ile, V/L/I), phenylalanine/tyrosine (Phe/Tyr, F/Y), lysine/arginine (Lys/Arg, K/R), alanine/valine (Ala/Val, A/V), glutamate/aspartate (Glu/Asp, E/D), and asparagine/glutamine (Asn/Gln, N/Q). In some embodiments, a conservative amino acid substitution can be a substitution of any native residue in a protein with alanine, as used in, for example, alanine scanning mutagenesis. In some embodiments, a conservative substitution is made that has a positive value in the PAM250 log-likelihood matrix disclosed in Gonnet, G. H. et al., 1992, Science 256:1443-1445, which is incorporated herein by reference in its entirety. In some embodiments, a substitution is a moderately conservative substitution wherein the substitution has a nonnegative value in the PAM250 log-likelihood matrix.

CONSERVATIVE AMINO ACID SUBSTITUTIONS

| For Amino Acid | Code | Replace With |
|---|---|---|
| Alanine | A | D-ala, Gly, Aib, β-Ala, Acp, L-Cys, D-Cys |
| Arginine | R | D-Arg, Lys, D-Lys, homo-Arg, D-homo-Arg, |
| | | Met, Ile, D-Met, D-Ile, Orn, D-Orn |
| Asparagine | N | D-Asn, Asp, D-Asp, Glu, D-Glu, Gln, D-Gln |
| Aspartic Acid | D | D-Asp, D-Asn, Asn, Glu, D-Glu, Gln, D-Gln |
| Cysteine | C | D-Cys, S-Me-Cys, Met, D-Met, Thr, D-Thr |
| Glutamine | Q | D-Gln, Asn, D-Asn, Glu, D-Glu, Asp, D-Asp |
| Glutamic Acid | E | D-Glu, D-Asp, Asp, Asn, D-Asn, Gln, D-Gln |
| Glycine | G | Ala, D-Ala, Pro, D-Pro, Aib, β-Ala, Acp |
| Isoleucine | I | D-Ile, Val, D-Val, AdaA, AdaG, Leu, D-Leu, |
| | | Met, D-Met |
| Leucine | L | D-Leu, Val, D-Val, AdaA, AdaG, Leu, D-Leu, |
| | | Met, D-Met |
| Lysine | K | D-Lys, Arg, D-Arg, homo-Arg, D-homo-Arg, |
| | | Met, D-Met, Ile, D-Ile, Orn, D-Orn |
| Methionine | M | D-Met, S-Me-Cys, Ile, D-Ile, Leu, D-Leu, |
| | | Val, D-Val |
| Phenylalanine | F | D-Phe, Tyr, D-Thr, L-Dopa, His, D-His, Trp, |
| | | D-Trp, Trans-3,4 or 5-phenylproline, AdaA, |
| | | AdaG, cis-3,4 or 5-phenylproline, Bpa, D-Bpa |
| Proline | P | D-Pro, L-I-thioazolidine-4-carboxylic acid, |
| | | D-or-L-1-oxazolidine-4-carboxylic acid |
| | | (Kauer, U.S. Pat. No. (4,511,390) |
| Serine | S | D-Ser, Thr, D-Thr, allo-Thr, Met, D-Met, |
| | | Met (O), D-Met (O), L-Cys, D-Cys |
| Threonine | T | D-Thr, Ser, D-Ser, allo-Thr, Met, D-Met, |
| | | Met (O), D-Met (O), Val, D-Val |
| Tyrosine | Y | D-Tyr, Phe, D-Phe, L-Dopa, His, D-His |
| Valine | V | D-Val, Leu, D-Leu, Ile, D-Ile, Met, D-Met, |
| | | AdaA, AdaG |

Control: As used herein, refers to the art-understood meaning of a “control” being a standard against which results are compared. Typically, controls are used to augment integrity in experiments by isolating variables in order to make a conclusion about such variables. In some embodiments, a control is a reaction or assay that is performed simultaneously with a test reaction or assay to provide a comparator. A “control” also includes a “control animal.” A “control animal” may have a modification as described herein, a modification that is different as described herein, or no modification (i.e., a wild-type animal). In one experiment, a “test” (i.e., a variable being tested) is applied. In a second experiment, the “control,” the variable being tested is not applied. In some embodiments, a control is a historical control (i.e., of a test or assay performed previously, or an amount or result that is previously known). In some embodiments, a control is or comprises a printed or otherwise saved record. A control may be a positive control or a negative control.

Determining, measuring, evaluating, assessing, assaying and analyzing: Determining, measuring, evaluating, assessing, assaying and analyzing are used interchangeably herein to refer to any form of measurement, and include determining if an element is present or not. These terms include both quantitative and/or qualitative determinations. Assaying may be relative or absolute. “Assaying for the presence of” can be determining the amount of something present and/or determining whether or not it is present or absent.

Dosage form: Those skilled in the art will appreciate that the term “dosage form” may be used to refer to a physically discrete unit of an agent (e.g., a therapeutic agent) for administration to a subject. Typically, each such unit contains a predetermined quantity of agent. In some embodiments, such quantity is a unit dosage amount (or a whole fraction thereof) appropriate for administration in accordance with a dosing regimen that has been determined to correlate with a desired or beneficial outcome when administered to a relevant population (i.e., with a therapeutic dosing regimen). Those of ordinary skill in the art appreciate that the total amount of a therapeutic composition or agent administered to a particular subject is determined by one or more attending physicians and may involve administration of multiple dosage forms.

Dosing regimen: Those skilled in the art will appreciate that the term “dosing regimen” may be used to refer to a set of unit doses (typically more than one) that are administered individually to a subject, typically separated by periods of time. In some embodiments, a given agent has a recommended dosing regimen, which may involve one or more doses. In some embodiments, a dosing regimen comprises a plurality of doses each of which is separated in time from other doses. In some embodiments, individual doses are separated from one another by a time period of the same length; in some embodiments, a dosing regimen comprises a plurality of doses and at least two different time periods separating individual doses. In some embodiments, all doses within a dosing regimen are of the same unit dose amount. In some embodiments, different doses within a dosing regimen are of different amounts. In some embodiments, a dosing regimen comprises a first dose in a first dose amount, followed by one or more additional doses in a second dose amount different from the first dose amount. In some embodiments, a dosing regimen comprises a first dose in a first dose amount, followed by one or more additional doses in a second dose amount same as the first dose amount In some embodiments, a dosing regimen is correlated with a desired or beneficial outcome when administered across a relevant population.

Engineered: In general, the term “engineered” refers to the aspect of having been manipulated by the hand of man. For example, a cell or organism is considered to be “engineered” if it has been manipulated so that its genetic information is altered (e.g., new genetic material not previously present has been introduced, for example by transformation, mating, somatic hybridization, transfection, transduction, or other mechanism, or previously present genetic material is altered or removed, for example by substitution or deletion mutation, or by mating protocols). As is common practice and is understood by those in the art, progeny of an engineered polynucleotide or cell are typically still referred to as “engineered” even though the actual manipulation was performed on a prior entity.

Functional: As used herein, a “functional” biological molecule is a biological molecule in a form in which it exhibits a property and/or activity by which it is characterized. A biological molecule may have two functions (i.e., bifunctional) or many functions (i.e., multifunctional).

Gene: As used herein, refers to a DNA sequence in a chromosome that codes for a product (e.g., an RNA product and/or a polypeptide product). In some embodiments, a gene includes coding sequence (i.e., sequence that encodes a particular product). In some embodiments, a gene includes non-coding sequence. In some particular embodiments, a gene may include both coding (e.g., exonic) and non-coding (e.g., intronic) sequence. In some embodiments, a gene may include one or more regulatory sequences (e.g., promoters, enhancers, etc.) and/or intron sequences that, for example, may control or impact one or more aspects of gene expression (e.g., cell-type-specific expression, inducible expression, etc.). For the purpose of clarity, we note that, as used in the present disclosure, the term “gene” generally refers to a portion of a nucleic acid that encodes a polypeptide or fragment thereof; the term may optionally encompass regulatory sequences, as will be clear from context to those of ordinary skill in the art. This definition is not intended to exclude application of the term “gene” to non-protein-coding expression units but rather to clarify that, in most cases, the term as used in this document refers to a polypeptide-coding nucleic acid.

Improve, increase, enhance, inhibit or reduce: As used herein, the terms “improve,” “increase,” “enhance,” “inhibit,” “reduce,” or grammatical equivalents thereof, indicate values that are relative to a baseline or other reference measurement. In some embodiments, a value is statistically significantly difference that a baseline or other reference measurement. In some embodiments, an appropriate reference measurement may be or comprise a measurement in a particular system (e.g., in a single individual) under otherwise comparable conditions absent presence of (e.g., prior to and/or after) a particular agent or treatment, or in presence of an appropriate comparable reference agent. In some embodiments, an appropriate reference measurement may be or comprise a measurement in comparable system known or expected to respond in a particular way, in presence of the relevant agent or treatment. In some embodiments, an appropriate reference is a negative reference; in some embodiments, an appropriate reference is a positive reference.

Isolated: As used herein, refers to a substance and/or entity that has been (1) separated from at least some of the components with which it was associated when initially produced (whether in nature and/or in an experimental setting), and/or (2) designed, produced, prepared, and/or manufactured by the hand of man. In some embodiments, an isolated substance or entity may be enriched; in some embodiments, an isolated substance or entity may be pure. In some embodiments, isolated substances and/or entities may be separated from about 10%, about 20%, about 30%, about 40%, about 50%, about 60%, about 70%, about 80%, about 90%, about 91%, about 92%, about 93%, about 94%, about 95%, about 96%, about 97%, about 98%, about 99%, or more than about 99% of the other components with which they were initially associated. In some embodiments, isolated agents are about 80%, about 85%, about 90%, about 91%, about 92%, about 93%, about 94%, about 95%, about 96%, about 97%, about 98%, about 99%, or more than about 99% pure. As used herein, a substance is “pure” if it is substantially free of other components. In some embodiments, as will be understood by those skilled in the art, a substance may still be considered “enriched”, “isolated” or even “pure”, after having been combined with certain other components such as, for example, one or more carriers or excipients (e.g., buffer, solvent, water, etc.); in such embodiments, percent isolation or purity of the substance is calculated without including such carriers or excipients. Those skilled in the art are aware of a variety of technologies for isolating (e.g., enriching or purifying) substances or agents (e.g., using one or more of fractionation, extraction, precipitation, or other separation).

Pharmaceutical composition: As used herein, the term “pharmaceutical composition” refers to a composition in which an active agent is formulated together with one or more pharmaceutically acceptable carriers. In some embodiments, the active agent is present in unit dose amount appropriate for administration in a therapeutic regimen that shows a statistically significant probability of achieving a predetermined therapeutic effect when administered to a relevant population. In some embodiments, a pharmaceutical composition may be specially formulated for administration in solid or liquid form, including those adapted for the following: oral administration, for example, drenches (aqueous or non-aqueous solutions or suspensions), tablets, e.g., those targeted for buccal, sublingual, and systemic absorption, boluses, powders, granules, pastes for application to the tongue, capsules, powders, etc. In some embodiments, an active agent may be or comprise a cell or population of cells (e.g., a culture, for example of an EES microbe); in some embodiments, an active agent may be or comprise an extract or component of a cell or population (e.g., culture) of cells. In some embodiments, an active agent may be or comprise an isolated, purified, or pure compound. In some embodiments, an active agent may have been synthesized in vitro (e.g., via chemical and/or enzymatic synthesis). In some embodiments, an active agent may be or comprise a natural product (whether isolated from its natural source or synthesized in vitro).

Pharmaceutically acceptable: As used herein, the term “pharmaceutically acceptable” which, for example, may be used in reference to a carrier, diluent, or excipient used to formulate a pharmaceutical composition as disclosed herein, means that the carrier, diluent, or excipient is compatible with the other ingredients of the composition and not deleterious to the recipient thereof.

Pharmaceutically acceptable carrier: As used herein, the term “pharmaceutically acceptable carrier” means a pharmaceutically-acceptable material, composition or vehicle, such as a liquid or solid filler, diluent, excipient, or solvent encapsulating material, involved in carrying or transporting the subject compound from one organ, or portion of the body, to another organ, or portion of the body. Each carrier must be is “acceptable” in the sense of being compatible with the other ingredients of the formulation and not injurious to the patient. Some examples of materials which can serve as pharmaceutically-acceptable carriers include: sugars, such as lactose, glucose and sucrose; starches, such as corn starch and potato starch; cellulose, and its derivatives, such as sodium carboxymethyl cellulose, ethyl cellulose and cellulose acetate; powdered tragacanth; malt; gelatin; talc; excipients, such as cocoa butter and suppository waxes; oils, such as peanut oil, cottonseed oil, safflower oil, sesame oil, olive oil, corn oil and soybean oil; glycols, such as propylene glycol; polyols, such as glycerin, sorbitol, mannitol and polyethylene glycol; esters, such as ethyl oleate and ethyl laurate; agar; buffering agents, such as magnesium hydroxide and aluminum hydroxide; alginic acid; pyrogen-free water; isotonic saline; Ringer's solution; ethyl alcohol; pH buffered solutions; polyesters, polycarbonates and/or polyanhydrides; and other non-toxic compatible substances employed in pharmaceutical formulations.

Prebiotic: As used herein, a “prebiotic” refers to an ingredient that allows or promotes specific changes, both in the composition and/or activity in the gastrointestinal microbiota that may (or may not) confer benefits upon the host. In some embodiments, a prebiotic can include one or more of the following: the prebiotic comprises a pome extract, berry extract and walnut extract.

Prevention: The term “prevention”, as used herein, refers to a delay of onset, and/or reduction in frequency and/or severity of one or more symptoms of a particular disease, disorder or condition. In some embodiments, prevention is assessed on a population basis such that an agent is considered to “prevent” a particular disease, disorder or condition if a statistically significant decrease in the development, frequency, and/or intensity of one or more symptoms of the disease, disorder or condition is observed in a population susceptible to the disease, disorder, or condition. In some embodiments, prevention may be considered complete, for example, when onset of a disease, disorder or condition has been delayed for a predefined period of time.

Reference: As used herein describes a standard or control relative to which a comparison is performed. For example, in some embodiments, an agent, animal, individual, population, sample, sequence or value of interest is compared with a reference or control agent, animal, individual, population, sample, sequence or value. In some embodiments, a reference or control is tested and/or determined substantially simultaneously with the testing or determination of interest. In some embodiments, a reference or control is a historical reference or control, optionally embodied in a tangible medium. Typically, as would be understood by those skilled in the art, a reference or control is determined or characterized under comparable conditions or circumstances to those under assessment. Those skilled in the art will appreciate when sufficient similarities are present to justify reliance on and/or comparison to a particular possible reference or control. In some embodiments, a reference is a negative control reference; in some embodiments, a reference is a positive control reference.

Risk: As will be understood from context, “risk” of a disease, disorder, and/or condition refers to a likelihood that a particular individual will develop the disease, disorder, and/or condition. In some embodiments, risk is expressed as a percentage. In some embodiments, risk is from 0, 1, 2, 3, 4, 5, 6, 7, 8, 9, 10, 20, 30, 40, 50, 60, 70, 80, 90 up to 100%. In some embodiments risk is expressed as a risk relative to a risk associated with a reference sample or group of reference samples. In some embodiments, a reference sample or group of reference samples have a known risk of a disease, disorder, condition and/or event. In some embodiments a reference sample or group of reference samples are from individuals comparable to a particular individual. In some embodiments, relative risk is 0,1, 2, 3, 4, 5, 6, 7, 8, 9, 10, or more.

Sample: As used herein, the term “sample” typically refers to an aliquot of material obtained or derived from a source of interest. In some embodiments, a source of interest is a biological or environmental source. In some embodiments, a source of interest may be or comprise a cell or an organism, such as a microbe, a plant, or an animal (e.g., a human). In some embodiments, a source of interest is or comprises biological tissue or fluid. In some embodiments, a biological tissue or fluid may be or comprise amniotic fluid, aqueous humor, ascites, bile, bone marrow, blood, breast milk, cerebrospinal fluid, cerumen, chyle, chime, ejaculate, endolymph, exudate, feces, gastric acid, gastric juice, lymph, mucus, pericardial fluid, perilymph, peritoneal fluid, pleural fluid, pus, rheum, saliva, sebum, semen, serum, smegma, sputum, synovial fluid, sweat, tears, urine, vaginal secretions, vitreous humour, vomit, and/or combinations or component(s) thereof. In some embodiments, a biological fluid may be or comprise an intracellular fluid, an extracellular fluid, an intravascular fluid (blood plasma), an interstitial fluid, a lymphatic fluid, and/or a transcellular fluid. In some embodiments, a biological fluid may be or comprise a plant exudate. In some embodiments, a biological tissue or sample may be obtained, for example, by aspirate, biopsy (e.g., fine needle or tissue biopsy), swab (e.g., oral, nasal, skin, or vaginal swab), scraping, surgery, washing or lavage (e.g., bronchioalveolar, ductal, nasal, ocular, oral, uterine, vaginal, or other washing or lavage). In some embodiments, a biological sample is or comprises cells obtained from an individual. In some embodiments, a sample is a “primary sample” obtained directly from a source of interest by any appropriate means. In some embodiments, as will be clear from context, the term “sample” refers to a preparation that is obtained by processing (e.g., by removing one or more components of and/or by adding one or more agents to) a primary sample. For example, filtering using a semi-permeable membrane. Such a “processed sample” may comprise, for example nucleic acids or proteins extracted from a sample or obtained by subjecting a primary sample to one or more techniques such as amplification or reverse transcription of nucleic acid, isolation and/or purification of certain components, etc.

Subject: As used herein, the term “subject” refers to an individual to which a provided treatment is administered. In some embodiments, a subject is animal. In some embodiments, a subject is a mammal, e.g., a mammal that experiences or is susceptible to a disease, disorder, or condition as described herein. In some embodiments, an animal is a vertebrate, e.g., a mammal, such as a non-human primate, (particularly a higher primate), a sheep, a dog, a rodent (e.g. a mouse or rat), a guinea pig, a goat, a pig, a cat, a rabbit, or a cow. Ins some embodiments, an animal is a non-mammal animal, such as a chicken, an amphibian, a reptile, or an invertebrate model C. elegans. In some embodiments, a subject is a human. In some embodiments, a patient is suffering from or susceptible to one or more diseases, disorders or conditions as described herein. In some embodiments, a patient displays one or more symptoms of a one or more diseases, disorders or conditions as described herein. In some embodiments, a patient has been diagnosed with one or more diseases, disorders or conditions as described herein. In some embodiments, the subject is receiving or has received certain therapy to diagnose and/or to treat a disease, disorder, or condition. In another embodiment, the subject is an experimental animal or animal substitute as a disease model.

Substantially: As used herein, refers to the qualitative condition of exhibiting total or near-total extent or degree of a characteristic or property of interest. One of ordinary skill in the biological arts will understand that biological and chemical phenomena rarely, if ever, go to completion and/or proceed to completeness or achieve or avoid an absolute result. The term “substantially” is therefore used herein to capture the potential lack of completeness inherent in many biological and chemical phenomena.

Symptoms are reduced: According to the present invention, “symptoms are reduced” when one or more symptoms of a particular disease, disorder or condition is reduced in magnitude (e.g., intensity, severity, etc.) and/or frequency. For purposes of clarity, a delay in the onset of a particular symptom is considered one form of reducing the frequency of that symptom.

Therapeutic regimen: A “therapeutic regimen”, as that term is used herein, refers to a dosing regimen whose administration across a relevant population may be correlated with a desired or beneficial therapeutic outcome.

Therapeutically effective amount: As used herein, is meant an amount that produces the desired effect for which it is administered. In some embodiments, the term refers to an amount that is sufficient, when administered to a population suffering from or susceptible to a disease, disorder, and/or condition in accordance with a therapeutic dosing regimen, to treat the disease, disorder, and/or condition. In some embodiments, a therapeutically effective amount is one that reduces the incidence and/or severity of, and/or delays onset of, one or more symptoms of the disease, disorder, and/or condition. Those of ordinary skill in the art will appreciate that the term “therapeutically effective amount” does not in fact require successful treatment be achieved in a particular individual. Rather, a therapeutically effective amount may be that amount that provides a particular desired pharmacological response in a significant number of subjects when administered to patients in need of such treatment. In some embodiments, reference to a therapeutically effective amount may be a reference to an amount as measured in one or more specific tissues (e.g., a tissue affected by the disease, disorder or condition) or fluids (e.g., blood, saliva, serum, sweat, tears, urine, etc.). Those of ordinary skill in the art will appreciate that, in some embodiments, a therapeutically effective amount of a particular agent or therapy may be formulated and/or administered in a single dose. In some embodiments, a therapeutically effective agent may be formulated and/or administered in a plurality of doses, for example, as part of a dosing regimen.

Treatment: As used herein, the term “treatment” (also “treat” or “treating”) refers to any administration of a therapy that partially or completely alleviates, ameliorates, relives, inhibits, delays onset of, reduces severity of, and/or reduces incidence of one or more symptoms, features, and/or causes of a particular disease, disorder, and/or condition. In some embodiments, such treatment may be of a subject who does not exhibit signs of the relevant disease, disorder and/or condition and/or of a subject who exhibits only early signs of the disease, disorder, and/or condition. Alternatively, or additionally, such treatment may be of a subject who exhibits one or more established signs of the relevant disease, disorder and/or condition. In some embodiments, treatment may be of a subject who has been diagnosed as suffering from the relevant disease, disorder, and/or condition. In some embodiments, treatment may be of a subject known to have one or more susceptibility factors that are statistically correlated with increased risk of development of the relevant disease, disorder, and/or condition.

### BRIEF DESCRIPTION OF THE DRAWING

FIG. 1 includes data showing that administration of Acetobacteraceae increased the lifespan of C. elegans. Panel (A) shows a lifespan assay on C. elegans animals administered either E. coli OP50, Gluconobacter oxydans, Acetobacter aceti or Gluconacetobacter hansenii. Compared to the animals administered E. coli OP50, C. elegans animals administered either Gluconobacter oxydans, Acetobacter aceti or Gluconacetobacter hansenii lived longer. Panel (B) includes a cumulative hazard plot of C. elegans animals administered either E. coli OP50, Gluconobacter oxydans, Acetobacter aceti or Gluconacetobacter hansenii. Panel (C) includes a Restricted Mean Lifespan (RMLS) of C. elegans animals administered either E. coli OP50, Gluconobacter oxydans, Acetobacter aceti or Gluconacetobacter hansenii. Panel (D) includes a statistical analysis of data shown in FIG. 1, panels (A)-(C).

FIG. 2 includes data showing that administration of Acetobacteraceae improved muscle function/activity. Panel (A) includes data obtained by measuring pharyngeal pumping in C. elegans animals administered either E. coli OP50, Gluconobacter oxydans, Acetobacter aceti or Gluconacetobacter hansenii. Compared to pharyngeal pumping rates of C. elegans animals administered E. coli OP50, the pumping rates in C. elegans animals administered either Gluconobacter oxydans, Acetobacter aceti or Gluconacetobacter hansenii were significantly higher in Day 6 and Day 12 C. elegans animals. The number of C. elegans animals scored are indicated on the top of each bar. Each bar shows Mean±s.d. Panel (B) includes data obtained by measuring body bends/minute in C. elegans animals administered either E. coli OP50, Gluconobacter oxydans, Acetobacter aceti or Gluconacetobacter hansenii. Compared to the number of body bends/minute in C. elegans animals administered E. coli OP50, the body bends/minute rates in animals administered either Gluconobacter oxydans, Acetobacter aceti or Gluconacetobacter hansenii were significantly higher in Day 6 and Day 12 animals. NS indicates no significant difference. The number of C. elegans animals scored are indicated on the top of each bar. Each bar shows Mean±s.d.

FIG. 3 includes data showing that administration of Acetobacteraceae improved stress resistance. Panel (A) includes data obtained from a UV resistance assay on C. elegans animals administered either E. coli OP50, Gluconobacter oxydans, Acetobacter aceti or Gluconacetobacter hansenii. Compared to UV-irradiated animals administered E. coli OP50, UV-irradiated C. elegans animals administered either Gluconobacter oxydans, Acetobacter aceti or Gluconacetobacter hansenii lived longer. Mean±s.d. for each measurement is plotted. Panel (B) includes data obtained from a thermotolerance assay on C. elegans animals administered either E. coli OP50, Gluconobacter oxydans, Acetobacter aceti or Gluconacetobacter hansenii. Compared to C. elegans animals shifted to 37° C. administered E. coli OP50, C. elegans animals shifted to 37° C. administered either Gluconobacter oxydans, Acetobacter aceti or Gluconacetobacter hansenii lived longer. Mean±s.d. for each measurement is plotted.

FIG. 4. includes data showing that administration of Acetobacteraceae decreased fat deposition. Compared to the animals administered E. coli OP50, C. elegans animals administered Gluconacetobacter hansenii had decreased fat levels as revealed by Oil Red O staining.

FIG. 5 includes data showing that prx-5 was needed for the G. hansenii-induced lifespan extension. Panel (A) includes data obtained from a lifespan assay on wildtype or prx-5(0) animals administered either E. coli OP50 or Gluconacetobacter hansenii. Compared to the C. elegans animals administered E. coli OP50, C. elegans animals administered either Gluconacetobacter hansenii lived longer. Lifespan curves of prx-5(0) animals administered either E. coli OP50 or Gluconacetobacter hansenii were similar. Panel (B) includes data obtained from a Restricted Mean Lifespan (RMLS) of wildtype or prx-5(0) C. elegans animals administered either E. coli OP50 or Gluconacetobacter hansenii. Panel (C) includes data obtained from a cumulative hazard plot of wildtype or prx-5(0) C. elegans animals administered either E. coli OP50 or Gluconacetobacter hansenii.

FIG. 6 includes data showing that tcer-1 and aak-2 were needed for the G. hansenii-induced lifespan extension. Panel (A) includes data obtained from a lifespan assay on wildtype or tcer-1(0) C. elegans animals administered either E. coli OP50 or Gluconacetobacter hansenii. Compared to the C. elegans animals administered E. coli OP50, C. elegans animals administered either Gluconacetobacter hansenii lived longer. Lifespan curves of tcer-1(0) C. elegans animals administered either E. coli OP50 or Gluconacetobacter hansenii are similar. Panel (B) includes data obtained from a Restricted Mean Lifespan (RMLS) of wildtype or tcer-1(0) C. elegans animals administered either E. coli OP50 or Gluconacetobacter hansenii. Panel (C) includes data obtained from a lifespan assay on wildtype or aak-2(0) C. elegans animals administered either E. coli OP50 or Gluconacetobacter hansenii. Compared to the C. elegans animals administered E. coli OP50, animals administered either Gluconacetobacter hansenii lived longer. Lifespan curves of aak-2(0) C. elegans animals administered either E. coli OP50 or Gluconacetobacter hansenii are similar. Panel (D) includes data obtained from a Restricted Mean Lifespan (RMLS) of wildtype or aak-2(0) C. elegans animals administered either E. coli OP50 or Gluconacetobacter hansenii.

FIG. 7 includes data showing that daf-16 was not required for the G. hansenii-induced lifespan extension. Panel (A) includes data obtained from a lifespan assay on wildtype or daf-16(0) C. elegans animals administered either E. coli OP50 or Gluconacetobacter hansenii. Panel (B) includes data obtained from a Restricted Mean Lifespan (RMLS) of wildtype or daf-16 (0) C. elegans animals administered either E. coli OP50 or Gluconacetobacter hansenii.

FIG. 8 includes data showing that hsf-1 was needed for the G. hansenii-induced thermotolerance phenotype. Panel (A) includes data showing G. hansenii administration does not affect heat shock protein expression. Panel (B) includes data obtained from a thermotolerance assay on wildtype or hsf-1(0) C. elegans animals administered either E. coli OP50 or Gluconacetobacter hansenii.

FIG. 9 includes data showing that an analysis of the genetic pathways required for G. hansenii-induced thermotolerance phenotype. Panel (A) includes data obtained from a thermotolerance assay on wildtype or tcer-1(0) C. elegans animals administered either E. coli OP50 or Gluconacetobacter hansenii. Panel (B) includes data obtained from a thermotolerance assay on wildtype or prx-5(0) C. elegans animals administered either E. coli OP50 or Gluconacetobacter hansenii. Panel (C) includes data obtained from a thermotolerance assay on wildtype or aak-2(0) C. elegans animals administered either E. coli OP50 or Gluconacetobacter hansenii.

### DETAILED DESCRIPTION

Aging is a complex process that affects numerous cellular processes and can lead to a wide variety of altered functions. In some instances, aging is accompanied by a gradual decline of tissue structure and cellular functions, which can lead to increased morbidity and mortality risk. Over the past century, human life expectancy has dramatically increased throughout the world (Beltran-Sanchez et al., 2015). Increased life expectancy poses new challenges in terms of healthcare and well-being of our aging population (Knickman and Snell, 2002). Chronic human ailments often associated with aging population, such as cardiovascular diseases, cancer, arthritis, diabetes, and neurodegenerative diseases, are increasing at an alarming rate throughout the world (Franceschi et al., 2018) (Lunenfeld and Stratton, 2013) (Frasca et al., 2017). Thus, a goal of aging research is to identify therapeutic interventions that can delay age-related decline in cellular function and promote longevity.

The present disclosure provides the recognition that microbial species present in the microbiome of a subject can impact the life span of the subject. The present disclosure provides the insight that certain microbes, particularly those in a microbiome (e.g., a human microbiome) can be modulated to modify the expected life span of a subject. For example, among other things, the present disclosure provides the recognition that the certain microbes can be administered to a subject and can extend the life span of the subject, and/or reduce or delay the onset of age-associated symptoms or conditions in a subject.

The present disclosure further provides that C. elegans are a powerful tool in determining which microbes of a microbiome can extend the life span of a subject, and/or reduce or delay the onset of age-associated symptoms or conditions in a subject. As such, the present disclosure provides technologies for identifying such microbes.

C. elegans

The free-living nematode C. elegans has been used extensively as a model system. C. elegans are inexpensive to cultivate, easy to physically manipulate, and has a multitude of genetic and molecular tools available for study. C. elegans are simple multicellular organisms: adults contain approximately 1,000 somatic cells yet have a variety of tissue types such as muscles, nerves, and intestinal cells. C. elegans have a short generation time, which allows for rapid experimentation. C. elegans generally progress from egg to larva to fertile adult in 3 days at room temperature. A single adult C. elegans can have between 300 and 1,000 progenies, which allows for a significant number of animals to be used and then quickly replenished in a relatively short amount of time. Due to the sexual dimorphism, C. elegans are useful for genetics. Self-fertilizing hermaphrodites can be maintained as homozygous mutations without the need for mating and males can be used for genetic crosses. C. elegans are transparent at every stage of their life cycle, which provides the ability to see inside the organism. This permits the observation of cellular events. It also permits the use of phosphorescent, luminescent, and fluorescent reporters. Manipulation of protein expression in C. elegans can also be performed using RNA-mediated interference (RNAi), which can allow for rapid assessment of gene function. Another advantage of using C. elegans a model system is the ability to freeze and recover the animals, thereby allowing long-term storage.

C. elegans can be genetically modified using a number of techniques to generate C. elegans strains. The sexual dimorphism of C. elegans allows for genetic manipulations to be performed with relative ease and according to know procedures. For example, if a strain needs to be propagated, single hermaphrodites can be used to self-fertilize and generate a population of offspring. Even if a mutation renders an animal unable to mate, it remains possible for a hermaphrodite to produce progeny. Another aspect of C. elegans reproduction that makes C. elegans an effective genetic tool is the animal's ability to cross males with hermaphrodites. For example, mating experiments allow genetic markers such as mutations causing visible phenotypes to be placed together in a single organism along with an unknown mutation in order to facilitate mapping of that mutation. Hermaphrodites make only a limited number of sperm and can typically have approximately 300 self-progeny. Mating increases the number of offspring produced by a single hermaphrodite to approximately 1,000 due to the addition of the male-produced sperm. The relatively large number of progeny coupled with the short life span of C. elegans allows for rapid and inexpensive analyses to be performed on the animals.

In addition to genetic modifications via reproduction, C. elegans can be genetically modified via injection of transgenes. Microinjection is an effective method for creating animals and for introducing various types of molecules directly to cells. For DNA transformation, one approach is to inject DNA into a distal arm of a C. elegans gonad. A distal germline of C. elegans contains a central core of cytoplasm that is shared by many germ cell nuclei. Therefore, DNA injected into a distal arm of a C. elegans gonad can be delivered to many progeny. Microinjection directly into oocyte nuclei can induce chromosomal integration of transgenes, but this technique can be more difficult to perform. C. elegans can also incorporate genetic material that is administered to them.

C. elegans are relatively simple to culture. C. elegans can be cultivated in either liquid culture or on the Nematode Growth Medium (NGM) agar plates in the presence of bacteria. It is possible to grow the animals in a chemically defined medium without the addition of bacteria, which can be useful because the components of a medium can be altered in order to study the nutrient or other chemical requirements of the animals. In some embodiments, C. elegans are grown on the agar plates. C. elegans can be grown on Nematode Growth Medium (NGM) agar plates. Bacteria can be spread on the NGM plates as a food source for the animals. For example, OP50, a leaky E. coli uracil auxotroph can be used. OP50 will grow slowly and provide nutrients for the animals without overgrowing them. Once the animals have eaten all of the food on a plate they will burrow into the agar and can be maintained on the “starved” plate for weeks at a time in a 15° C. incubator. The animals can be transferred to an agar plate with fresh bacteria by either cutting and moving a small block of agar from the starved plate with a sterile instrument such as a micropipette tip, or washing the animals off the surface of the plate with sterile water, or by picking one or more individuals onto a fresh plate, which will cause the C. elegans to reemerge. At any time, C. elegans can be cryogenically preserved. C. elegans prefer to grow between 15° C. and 25° C., but the temperature can vary depending on the strain of C. elegans and conditions being tested. In some embodiments, a C. elegans culture can be cultured at a temperature of at least 5° C., at least 10° C., at least 15° C., at least 20° C., at least 25° C., at least 30° C., at least 35° C., or at least 40° C. In some embodiments, a C. elegans culture can be cultured at a temperature of at most 65° C., at most 60° C., at most 55° C., at most 50° C., at most 55° C., at most 40° C., at most 35° C., at most 30° C., at most 25° C., or at most 20° C. Standard protocols for C. elegans manipulation and culture are known, e.g., as described by Stiernagle T. Maintenance of C. elegans. Wormbook, ed. The C. elegans Research Community, WormBook. (Feb. 11, 2006), which is incorporated herein by reference.

The bacterivorous nematode Caenorhabditis elegans is an outstanding model organism aging studies because of a short lifespan (˜15 days). C. elegans is a powerful model for studying genetic pathways that modulate the aging process (Knickman and Snell, 2002) (Johnson, 2003) (Antebi, 2007) (Wilkinson et al., 2012). C. elegans is well-suited for forward and reverse genetic approaches as well as for identifying and characterizing small-molecule compounds that influence aging (Antebi, 2007) (Collins et al., 2006) (Denzel et al., 2019) (Arey and Murphy, 2017). Studies in C. elegans have discovered conserved genetic pathways that modulate aging and which correspond to pathways involved in human longevity (Bitto et al., 2015) (Collins et al., 2006) (Arey and Murphy, 2017) (Finch and Ruvkun, 2001). These include the insulin/IGF-1 like signaling (IIS) pathway (Tissenbaum and Ruvkun, 1998) (Kenyon, 2011), the target of rapamycin (TOR) (Robida-Stubbs et al., 2012) (Johnson et al., 2013), Nrf2/Antioxidant stress response pathway (Blackwell et al., 2015), TGF beta signaling (Kaplan et al., 2015) (Luo et al., 2010), Sirtuins (Dang, 2014) (Guarente, 2007) (Longo and Kennedy, 2006), autophagy(Gelino et al., 2016) (Hansen et al., 2008) (Chang et al., 2017), and the AMP-activated protein kinase (AMPK) pathways (Burkewitz et al., 2014) (Curtis et al., 2006) (Onken and Driscoll, 2010). In view of similarities between animals, mechanistic pathways that influence lifespan may be conserved throughout animal evolution.

Interventions that have been demonstrated to delay aging and extend lifespan in C. elegans, include perturbations in nutrient sensing, dietary restriction, mutations affecting mitochondrial metabolism, mutations affecting ribosomal function and drugs such as rapamycin (Kapahi et al., 2017) (Finch and Ruvkun, 2001) (Srivastava, 2017) (Pan and Finkel, 2017) (Bansal et al., 2015) (Kenyon, 2005) (Wilkinson et al., 2012). Thus, C. elegans can represent a powerful model to identify and characterize interventions that promote healthy aging and may be beneficial in humans (Johnson, 2003).

Several recent studies implicate a major role for the human gut microbiome in regulating various aspects of human development including aging (Vaiserman et al., 2017) (Zapata and Quagliarello, 2015) (Bischoff, 2016). The microbiome directly affects host development by, among other things, providing nutrients and essential metabolic compounds (Choi et al., 2018). Dramatic changes in the microbiome composition were observed between infants and adults, and between middle-aged and older adults (Choi et al., 2018)(An et al., 2018)(Claesson et al., 2012)(Kim and Jazwinski, 2018) (Gerber, 2014) (Maffei et al., 2017). Also, changes in microbiome composition have been suggested as an important factor in several age-related conditions, including metabolic syndrome and cancer (Tilg and Kaser, 2011). Although changes in microbiome composition are likely to result in altered microbial metabolism, how these changes affect aging is not understood. The majority of metabolites in human plasma are microbe-derived and the gut microbiome is a likely source. Whether these microbiome-derived metabolic factors might affect aging process is not known.

Technologies provided in the present disclosure can be used to for the identification of microbes, extracts, or microbiome-derived components (e.g., factors, metabolites, etc.) that modulate the aging processes, define the conserved signaling pathways through which these microbes or microbiome-derived factors influence aging and to develop novel therapeutics based on these factors for beneficial impacts on overall human health in old age. Since both C. elegans and bacteria are genetically tractable, it is possible to use technologies described herein to assess how diet affects aging in an unbiased fashion.

C. elegans is a bacterivore nematode that feeds on various bacterial species growing on rotting fruits and vegetation. Many of these microbes also colonize the C. elegans gut to serve as its microbiome. In the laboratory, C. elegans are administered exclusively E. coli OP50. E. coli act as nutrition for the animal, providing essential nutrients that the nematode cannot synthesize de novo. C. elegans is emerging as a powerful model to study the effects of diet on aging because it is possible to easily replace the standard diet of E. coli with other microbes (MacNeil and Walhout, 2013). Recent studies in C. elegans suggest that bacteria-derived diffusible metabolites can directly impact C. elegans aging (Ezcurra, 2018) (Smith et al., 2008). Animals administered E. coli mutants that were unable to synthesize coenzyme Q were found to live longer (Jonassen et al., 2001). The C. elegans lifespan extending effect of Metformin (widely used for diabetes treatment) was found to be due to alterations in bacterial folate and methionine metabolism (Cabreiro et al., 2013) (Onken and Driscoll, 2010). Genetic or pharmacological inhibition of E. coli folate synthesis leads to an increase in C. elegans lifespan (Maynard et al., 2018). Strain-specific effects of E. coli on C. elegans lifespan were found to be due to structural differences in Lipopolysaccharides (Maier et al., 2010). Bacillus subtilis-derived NO was found to extend lifespan via modulation of the DAF-16/FOX0 and heat shock factor 1 (HSF-1) pathways (Donato et al., 2017). Probiotic bacteria such as Lactobacillus and Bifidobacterium can enhance immunity and extend lifespan in C. elegans (Zhao et al., 2013)(Fasseas et al., 2013) (Grompone et al., 2012) (Komura et al., 2013) (Martorell et al., 2016) (Sugawara and Sakamoto, 2018) (Zhao et al., 2017). Studies in C. elegans have also uncovered that the effect of genetic mutations on lifespan can depend upon the type of specific bacterial diet (Maier et al., 2010) (Brooks et al., 2009) (Heintz and Mair, 2014). TOR complex-2-specific factor Rictor mutants are short-lived when grown on E. coli OP50 bacteria but long-lived when cultured on E. coli HT115 (Soukas et al., 2009). C. elegans alh-6 (aldehyde dehydrogenase gene) mutants are short-lived when cultured on E. coli OP50 but not when cultured in HT115 (Pang and Curran, 2014). The underlying mechanism(s) involved in these differences are not known; however, it is possible that metabolites or signals produced by these E. coli strains could be one of the contributing factors. In summary, these studies represent the beginning of an era of exploration into how the microbiome influence host longevity. Studies from several labs have identified a core set of microbes that constitute the natural microbiome of C. elegans (Dirksen et al., 2016) (Félix and Braendle, 2010). Animals sampled directly from their native habitats carry a variety of bacteria, dominated by Proteobacteria, Bacteroidetes, Firmicutes, and Actinobacteria (Samuel et al., 2016). A C. elegans microbiome was found to be distinct from its natural habitat suggesting a selective or preferential gating of microbes. Although the effects of feeding individual bacterial species of the C. elegans microbiome on animal development have been investigated, a systematic analysis of the effects of the microbiome on C. elegans aging has not previously been conducted.

Compositions

The present disclosure provides compositions comprising at least one bacterial strain or extract(s) or component(s) thereof, and an excipient. While the present disclosure provides exemplary microbes (e.g., bacterial strains) that affect aging, the present disclosure also provides methods for identifying additional microbes that may be used in accordance with the compositions and methods described herein.

In some embodiments, at least one bacterial strain comprises a Gluconobacter spp., Acetobacter spp., Gluconoacaetobacter spp., Acidomonas spp., Ameyamaea spp., Asaia spp., Granulibacter spp., Kozakia spp., Neoasaia spp., Neokomagataea spp., Saccharibacter spp., Swaminathania spp., Tanticharoenia spp., or a combination thereof In some embodiments, at least one bacterial strain comprises Gluconobacter albidus, Gluconobacter cerinus, Gluconobacter frateruii, Gluconobacter japonicus, Gluconobacter kondonii, Gluconobacter nephelii, Gluconobacter oxydans, Gluconoacetobacter diazotrophicus, Gluconoacetobacter hansenii, Gluconoacetobacter saccharivorans, Acetobacter aceti, Acetobacter malorum, or a combination thereof. In some embodiments, at least one bacterial strain comprises Gluconacetobacter hansenii, Gluconobacter oxydans, Acetobacter aceti, or a combination thereof. In some embodiments, at least one bacterial strain comprises Gluconacetobacter hansenii.

In some embodiments, a composition comprises at least one bacterial strain. In some embodiments, a composition comprises at least 2 bacterial strains, at least 3 bacterial strains, at least 4 bacterial strains, at least 5 bacterial strains, at least 6 bacterial strains, at least 7 bacterial strains, at least 8 bacterial strains, at least 9 bacterial strains, at least 10 bacterial strains, at least 15 bacterial strains, or at least 20 bacterial strains. In some embodiments, a composition comprises at most 100 bacterial strains, at most 90 bacterial strains, at most 80 bacterial strains, at most 70 bacterial strains, at most 60 bacterial strains, at most 50 bacterial strains, at most 40 bacterial strains, at most 30 bacterial strains, at most 20 bacterial strains, at most 10 bacterial strains, or at most 5 bacterial strains.

In some embodiments, extract(s) of at least one bacterial strain comprise one or more extracts of the at least one bacterial strain. In some embodiments, component(s) of at least one bacterial strain comprise one or more extracts of the at least one bacterial strain. Accordingly, comprising at least one bacterial strain or extract(s) or component(s) thereof as described herein can include, e.g., two extracts from Gluconobacter oxydans, a component from Acetobacter aceti, and Gluconacetobacter hansenii.

Compositions described herein can include an excipient. In some embodiments, an excipient is or comprises an inactive (e.g., non-biologically active) agent. An excipient may be included in a composition, for example, to provide or contribute to a desired consistency or stabilizing effect. In some embodiments, excipients may include, for example, starch, glucose, lactose, sucrose, gelatin, malt, rice, flour, chalk, silica gel, sodium stearate, glycerol monostearate, talc, sodium chloride, dried skim milk, glycerol, propylene, glycol, water, or ethanol.

In some embodiments, compositions for use in accordance with the present disclosure are pharmaceutical compositions, e.g., for administration (e.g., oral administration) to a mammal (e.g., a human). Pharmaceutical compositions typically include an active agent (e.g., individual microbial strains or combinations of microbial strains), and an excipient. An excipient can be a pharmaceutically acceptable carrier, for instance saline, solvents, dispersion media, coatings, antibacterial and antifungal agents, isotonic and absorption delaying agents, and the like, compatible with pharmaceutical administration.

In some embodiments, a composition or a pharmaceutical composition for use in accordance with the present disclosure may include and/or may be administered in conjunction with, one or more supplementary active compounds; in certain embodiments, such supplementary active agents can include ginger, curcumin, probiotics (e.g, probiotic strains of one or more of the following genera: Lactobacillus, Bifidobacterium, Saccharomyces, Enterococcus, Streptococcus, Pediococcus, Leuconostoc, Bacillus, and/or Escherichia coli (see Fijan, Int J Environ Res Public Health. 2014 May; 11(5): 4745-4767, which is incorporated herein by reference); prebiotics (nondigestible food ingredients that help support growth of probiotic microbes, e.g., fructans such as fructooligosaccharides (FOS) and inulins, galactans such as galactooligosaccharides (GOS), dietary fibers such as resistant starch, pectin, beta-glucans, and xylooligosaccharides (Hutkins et al., Curr Opin Biotechnol. 2016 Feb; 37: 1-7, which is incorporated herein by reference) and combinations thereof.

Compositions or pharmaceutical compositions are typically formulated to be compatible with their intended route of administration. Examples of routes of administration include oral administration. Methods of formulating suitable compositions have been reported, see, e.g., Remington: The Science and Practice of Pharmacy, 21st ed., 2005; and the books in the series Drugs and the Pharmaceutical Sciences: a Series of Textbooks and Monographs (Dekker, N.Y.). Oral compositions generally include an inert diluent or an edible carrier. To give but a few examples, in some embodiments, an oral formulation may be or comprise a syrup, a liquid, a tablet, a troche, a gummy, a capsule, e.g., gelatin capsules, a powder, a gel, a film, etc.

In some embodiments, compatible binding agents, and/or adjuvant materials can be included as part of a composition (e.g., pharmaceutical composition). In some particular embodiments, a composition can contain, e.g., any one or more of the following inactive ingredients, or compounds of a similar nature: a binder such as microcrystalline cellulose, gum tragacanth or gelatin; an excipient such as starch or lactose, a disintegrating agent such as alginic acid, Primogel, or corn starch; a lubricant such as magnesium stearate or Sterotes; a glidant such as colloidal silicon dioxide; a sweetening agent such as sucrose or saccharin; or a flavoring agent such as peppermint, methyl salicylate, or orange flavoring. In some embodiments, compositions can be taken as-is or sprinkled onto or mixed into a food or liquid (such as water). In some embodiments, a composition that may be administered to subjects as described herein may be or comprise an ingestible item (e.g., a food or drink) that comprises (e.g., is supplemented) with an individual microbial strain or combinations of microbial strains (e.g., from a mammalian microbiome), extracts thereof, and/or components thereof.

In some embodiments, a food can be or comprise one or more of bars, candies, baked goods, cereals, salty snacks, pastas, chocolates, and other solid foods, as well as liquid or semi-solid foods including yogurt, soups and stews, and beverages such as smoothies, shakes, juices, and other carbonated or non-carbonated beverages. In some embodiments, foods are prepared by a subject by mixing in individual microbial strains or combinations of microbial strains (e.g., from a mammalian microbiome), extracts thereof, and/or components thereof.

Compositions can be included in a kit, container, pack, or dispenser, together with instructions for administration or for use in a method described herein.

In some embodiments, at least one microbial (e.g., bacterial) strain that have been killed (e.g., heat killed). Alternatively, in some embodiments, at least one microbial (e.g., bacterial) strains may include cells that are viable or alive.

In some embodiments, methods of treatment as described herein involve administering at least one viable or living microbial (e.g., bacterial) strain. In some such embodiments, at least one viable or living microbial (e.g., bacterial) strain is administered according to a regimen that achieves population of the subject's microbiome with administered cells.

In some embodiments, at least one microbial (e.g., bacterial) strain as described herein comprises and/or is formulated through use of one or more cell cultures and/or supernatants or pellets thereof, and/or a powder formed therefrom.

In some embodiments, a pharmaceutical composition provided herein can promote the colonization of at least one microbial (e.g., bacterial) strain, particularly microbial strain(s) that have been identified, characterized, or assessed as extending life span, or reducing or delaying the onset of at least one age-associated symptom or condition in a subject. In some embodiments, a pharmaceutical composition provided herein can promote the colonization of at least one microbial (e.g., bacterial) strain, particularly microbial strain(s) that have been identified, characterized, or assessed as extending life span, or reducing or delaying the onset of at least one age-associated symptom or condition in a subject.

In some embodiments, a pharmaceutical composition is tailored to a specific mammal (e.g., a specific human subject) based on that mammal's (e.g., human's) microbiome. In some embodiments, a pharmaceutical composition is specific for a microbiome of a mammalian subject (e.g., human). In some embodiments, a pharmaceutical composition is specific for microbiomes of a population of mammals (e.g., humans). Populations of mammals can include, but are not limited to: families, mammals in the same regional location (e.g., neighborhood, city, state, or country), mammals with the same disease or condition, mammals of a particular age or age range, mammals that consume a particular diet (e.g., food, food source, or caloric intake).

In some embodiments, a composition described herein is formulated for oral administration. In some embodiments, a composition is a food, a beverage, a feed composition, or a nutritional supplement. In some embodiments, a composition is a liquid, syrup, tablet, troche, gummy, capsule, powder, gel, or film. In some embodiments, a composition is a pharmaceutical composition. In some embodiments, a composition is an enteric-coated formulation.

Compositions described herein can affect aging or sign of aging. As discussed above, a model system by which to characterize the ability of a microbe (e.g., bacterial strain) in a composition can be C. elegans. For example, in some embodiments, at least one bacterial strain or extract(s) or component(s) thereof is characterized in that, when administered to a C. elegans culture comprising C. elegans animals, an average life span of the C. elegans animals in the C. elegans culture is extended by at least 20%, at least 25%, at least 30%, at least 35%, at least 40%, at least 45%, or at least 50%, as compared to that of C. elegans animals in a comparable C. elegans culture without administration of the at least one bacterial strain or extract or component thereof. A life span is a time period between the birth of a subject and the death of a subject. An average life span can be the average time period between the birth and the death of a plurality of subjects (e.g., C. elegans, mammals, humans).

In some embodiments, at least one bacterial strain or extract(s) or component(s) thereof is characterized in that, when administered to a C. elegans culture comprising C. elegans animals, average pharyngeal pumping activity of the C. elegans animals in the C. elegans culture is increased by at least 20%, at least 25%, at least 30%, at least 35%, at least 40%, at least 45%, or at least 50%, as compared to that of C. elegans animals in a comparable C. elegans culture without administration of the at least one bacterial strain or extract(s) or component(s) thereof. Pharyngeal pumping activity can be measured, e.g., by counting grinder movements, e.g., a single contraction and relaxation of a C. elegans corpus and/or terminal bulb. In some embodiments, pharyngeal pumping activity can be measure in pumps (or grinder movements) per minute (ppm). An average pharyngeal pumping activity can be the average number of pumps (e.g., per minute) of a plurality of subjects (e.g., C. elegans, mammals, humans).

In some embodiments, at least one bacterial strain or extract(s) or component(s) thereof is characterized in that, when administered to a C. elegans culture comprising C. elegans animals, average locomotion rate of the C. elegans animals in the C. elegans culture is increased by at least 20%, at least 25%, at least 30%, at least 35%, at least 40%, at least 45%, or at least 50%, as compared to that of C. elegans animals in a comparable C. elegans culture without administration of the at least one bacterial strain or extract(s) or component(s) thereof. In some embodiments, a locomotion rate can be calculated by animal bends per minute. An average locomotion rate can be the average number of animal bends (e.g., per minute) of a plurality of subjects (e.g., C. elegans, mammals, humans).

In some embodiments, at least one bacterial strain or extract(s) or component(s) thereof is characterized in that, when administered to a C. elegans culture comprising C. elegans animals, fertility of the C. elegans animals in the C. elegans culture is decreased by at least 20%, at least 25%, at least 30%, at least 35%, at least 40%, at least 45%, or at least 50%, as compared to that of C. elegans animals in a comparable C. elegans culture without administration of the at least one bacterial strain or extract(s) or component(s) thereof. In some embodiments, fertility can be determined by the number of reproductive events (e.g., births) that occur. In some embodiments, fertility can be determined by the number of progeny. An average fertility rate can be the average number of reproductive events or the average number of progeny for a plurality of animals, e.g., C. elegans.

In some embodiments, at least one bacterial strain or extract(s) or component(s) thereof is characterized in that, when a C. elegans culture comprising C. elegans animals is exposed to Ultra Violet irradiation, average survival time of the C. elegans animals in the C. elegans culture to which the at least one bacterial strain or extract(s) or component(s) thereof has been administered is increased by at least 20%, at least 25%, at least 30%, at least 35%, at least 40%, at least 45%, or at least 50%, as compared to that of C. elegans animals in a comparable C. elegans culture without administration of the at least one bacterial strain or extract(s) or component(s) thereof. In some embodiments, survival time is measured from the time the animal is exposed to UV irradiation until the time the animal dies.

In some embodiments, at least one bacterial strain or extract(s) or component(s) thereof is characterized in that, when a C. elegans culture comprising C. elegans animals is exposed to an elevated temperature, average survival time of C. elegans animals in the C. elegans culture to which the at least one bacterial strain or extract(s) or component(s) thereof has been administered is increased by at least 20%, at least 25%, at least 30%, at least 35%, at least 40%, at least 45%, or at least 50%, as compared to that C. elegans animals in of a comparable C. elegans culture without administration of the at least one bacterial strain or extract(s) or component(s) thereof. In some embodiments, an elevated temperature is at least 37° C., at least 40° C., at least 45° C., at least 50° C., at least 55° C., at least 60° C., at least 65° C., at least 70° C., at least 75° C., or at least 80° C. In some embodiments, an elevated temperature is 50° C.-65° C., 65° C.-80° C., or 80° C.-120° C. In some embodiments, survival time is measured from the time the elevated temperature is reached until the time the animal dies.

In some embodiments, at least one bacterial strain or extract(s) or component(s) thereof is characterized in that, when administered to a C. elegans culture comprising C. elegans animals, average amount of intestinal fat observed in the C. elegan animals in the C. elegans culture is decreased by at least 20%, at least 25%, at least 30%, at least 35%, at least 40%, at least 45%, or at least 50%, as compared to that of C. elegans animals in a comparable C. elegans culture without administration of the at least one bacterial strain or extract(s) or component(s) thereof. In some embodiments, an amount of intestinal fat is determined by visual observation following staining, e.g., Oil red O staining. In some embodiments, the area stained by, e.g., Oil red O stain, can be measured.

In some embodiments, C. elegans animals are adult C. elegans animals. In some embodiments, C. elegans animals are at least 5 days old.

Methods

The present disclosure provides the recognition that compositions described herein can be useful in extending life span, or reducing or delaying an age-associated symptom or condition in a subject. The present disclosure provides methods comprising administering a composition described herein to a subject a composition. As discussed above, a composition can be formulated to be compatible with their intended route of administration.

In some embodiments, a method is a method of extending lifespan of a subject. In some embodiments, the life span of a subject is extended by at least 20%, at least 25%, at least 30%, at least 35%, at least 40%, at least 45%, or at least 50%, as compared to that of a comparable subject without administration of the composition.

In some embodiments, a method is a method of reducing or delaying the onset of at least one age-associated symptom or condition in a subject. In some embodiments, at least one age-associated symptom or condition is reduced or delayed in a subject by at least 20%, at least 25%, at least 30%, at least 35%, at least 40%, at least 45%, or at least 50%, as compared to that of a comparable subject without administration of the composition. In some embodiments, at least one age-associated symptom or condition is or comprises a decline in muscle and/or neuromuscular function of a subject. In some embodiments, at least one age-associated symptom or condition is or comprises dysregulation of lipid metabolism. In some embodiments, at least one age-associated symptom or condition is or comprises, e.g., a level of mitosis, organ function, organ wall thickness, variability in core body temperature, bone density, a level of peristalsis, retinal thickness, eardrum thickness, hearing loss, vision loss, or a combination thereof.

In some embodiments, a subject is at least 30 years old, at least 35 years old, at least 40 years old, at least 45 years old, at least 50 years old, at least 55 years old, at least 60 years old, at least 65 years old, at least 70 years old, or at least at least 75 years old. In some embodiments, a subject is an elderly subject. A subject could be younger than 30 years old, however, if, e.g., the subject suffers from a disease or condition associate with premature aging.

In some embodiments, a method is a method of treating a subject who has or is at risk of developing a disease or disorder associated with premature aging. In some embodiments, a disease or disorder is Bloom syndrome, Bockayne Syndrome, Hutchinson-Gilford progeria syndrome, mandibuloacral dysplasia with type A lipodystrophy, progeria, progeroid syndrome, Rothmund-Thomson syndrome, Seip syndrome, or Werner syndrome.

In some embodiments, a subject is a mammal. In some embodiments, a mammal is a non-human primate (e.g, a higher primate), a sheep, a dog, a rodent (e.g., a mouse or rat), a guinea pig, a goat, a pig, a cat, a rabbit, or a cow. In some embodiments, a mammal is a human.

In some embodiments, a method comprises administering comprises administering a sufficient amount of the microbe to colonize the subject's microbiome.

Assessing Biological Impact

The present disclosure provides the insight that C. elegans can be used to identify, characterize, or assess microbial strain(s) of a mammalian microbiome for an ability to extend the life span of a subject, or reduce or delay an age-associated symptom and/or an age-associated condition by contacting the microbial strain(s) (e.g., feeding the microbial strain(s) to, administering to) C. elegans. To determine whether a microbial strain or combination of microbial strains extend the life span, or reduce or delay an age-associated symptom and/or an age-associated condition of C. elegans can be observed, measured, or assessed in different samples that have been contacted with the microbial strain or combination of microbial strains. As just a few examples, parameters can include muscle function/activity, e.g., locomotion or bending, reproduction, stress resistance, lipid metabolism, or a combination thereof. In some embodiments, parameters can include, alone or in addition to those previously listed, genetic mutations (e.g., the presence of SNPs, deletions, additions, inversions, or repeats in DNA), transcript levels, protein levels, metabolite levels, lipid levels, carbohydrate levels, protein (e.g., enzyme) activity levels can be observed, measured, or assessed to determine whether a microbial strain or combination of microbial strains affects the life span of a subject, or reduce or delay an age-associated symptom and/or an age-associated condition of a C. elegans.

In some embodiments, methods described herein utilize a first sample and a second sample. In some embodiments, a first sample is a reference sample. In some embodiments, a reference sample can be a culture of C. elegans contacted with (e.g., administered or fed), e.g., OP50. In some embodiments, a reference sample can be a culture of C. elegans contacted with (e.g., administered or fed) a microbial strain or combination of microbial strains from a microbiome of a healthy individual. In some embodiments, a reference sample can be a culture of C. elegans contacted with (e.g., administered or fed) a microbial strain or combination of microbial strains from a microbiome of an individual obtained at a first time point.

In some embodiments, a second sample can be a test sample. In some embodiments, a test sample can be a culture of C. elegans contacted with (e.g., administered or fed) an individual microbial strain or a combination of microbial strains from a mammalian microbiome, e.g., a human microbiome. In some instances, a human microbiome is a microbiome of a human suffering from or at risk of a disease or condition, e.g., a disease or condition associated with premature aging or delayed aging. In some embodiments, a test sample can be a culture of C. elegans contacted with (e.g., administered or fed) a microbial strain or combination of microbial strains from a microbiome of an individual obtained at a second time point (e.g., an aged subject).

In some embodiments, methods described herein comprise comparing one or more parameters obtained from a test sample with one or more parameters obtained from a reference sample. In some embodiments, by comparing one or more parameters obtained from a test sample with one or more parameters obtained from a reference sample, it can be determined that an individual microbial strain or a combination of microbial strains from a microbiome affect the life span, or reduce or delay an age-associated symptom and/or an age-associated condition of a C. elegans culture. In some embodiments, by comparing one or more parameters obtained from a test sample with one or more parameters obtained from a reference sample, it can be determined that an individual microbial strain or a combination of microbial strains from a microbiome extend the life span, or reduce or delay an age-associated symptom and/or an age-associated condition of the cultured C. elegans.

C. elegans and methods using C. elegans provided herein can be useful in assessing, characterizing, or identifying microbial strains of a microbiome that affect life span, or reduce or delay an age-associated symptom and/or an age-associated condition. The present disclosure provides the recognition that C. elegans and methods using C. elegans provided herein can be used to define and/or characterize a microbial signature associated with the life span of a subject, or one or more age-associated symptoms and/or an age-associated conditions.

The present disclosure also provides the recognition that C. elegans and methods using C. elegans provided herein can be used to monitor age progression.

The present disclosure also provides the insight that C. elegans and methods using C. elegans provided herein can be used to tailor therapeutics (e.g., therapies, nutraceuticals, and/or probiotics) to an individual patient. In some cases, microbial strains within an individual can be assessed, characterized, or identified to determine if they have an effect on an age-associated symptom and/or an age-associated condition. Based on the results, the individual can be administered one or more microbial strains to adjust the microbial strains (and/or component or compound thereof) in their microbiome. In some instances, this will affect aging of the individual. For example, if an individual is determined to have a relatively low amount of one or more microbial strains that have been determined to extend life spans, administration of the one or more microbial strains can extend the life span of the individual.

Among other things, the present disclosure provides technologies for assessing one or more microbes for usefulness as described herein. In some embodiments, technologies for identifying and/or characterizing microbes as described herein may involve comparisons of observations or measurements made on C. elegans administered the microbes with an appropriate reference (e.g., with a positive control references and/or with a negative control reference). In some embodiments, a reference may be or comprise a historical reference; in some embodiments, a reference may be or comprise a contemporaneous reference.

### EXAMPLES

The following examples are provided so as to describe to the skilled artisan how to make and use methods and compositions described herein, and are not intended to limit the scope of the present disclosure.

A library of ˜30 bacterial species was screened for their effect on C. elegans lifespan. Bacterial species were chosen based on an abundant representation in 16s RNA sequencing studies. In this screen, 3 bacterial species were identified that significantly increased the lifespan of wildtype C. elegans animals. Such a screen can be repeated with additional microbial species to determine if such species affect lifespan according to technologies described herein.

### Example 1: Administration of Acetobacteraceae Increased the Lifespan of C. elegans

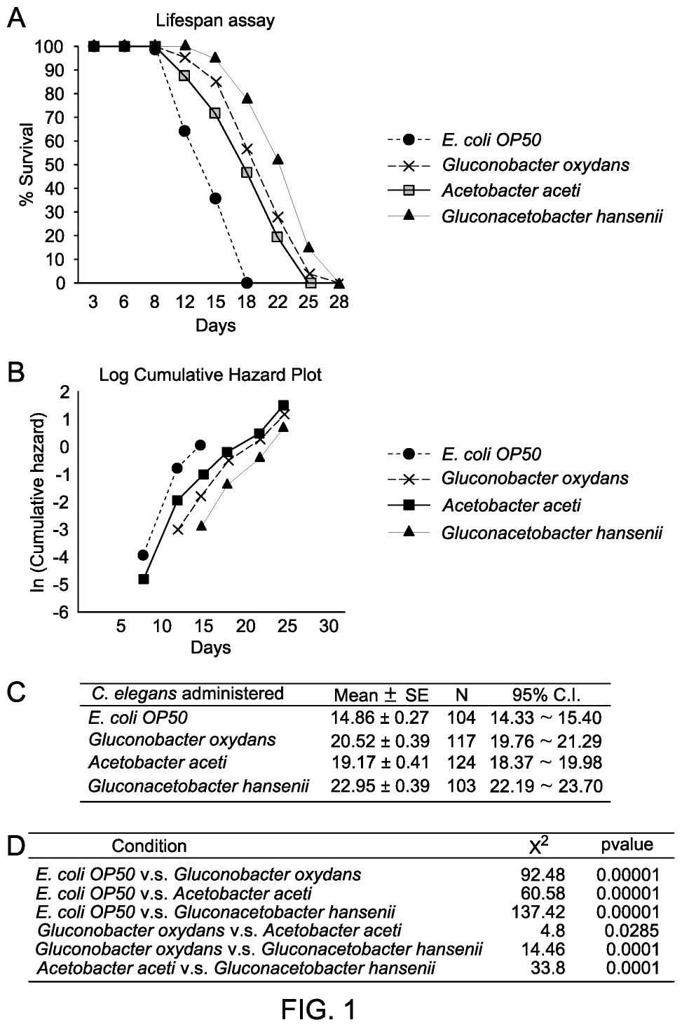

C. elegans (N2) longevity assays were performed on NGM plates seeded with either E. coli OP50, G. oxydans, A. aceti, or G. hansenii (FIG. 1, panels A-D). C. elegans animals administered (e.g. fed) either G. oxydans, A. aceti, or G. hansenii exhibited a significantly (P <0.00001) increased lifespan compared to animals administered E. coli OP50 (FIG. 1, panel A). A cumulative hazard plot analysis generated using the OASIS 2 platform (Han et al., 2016) showed that the hazard rates were different between animals administered E. coli OP50 and either G. oxydans, A. aceti, or G. hansenii (FIG. 1, panel B). Hazard plots of animals administered G. hansenii compared with animals administered either G. oxydans or A. aceti was slightly different from each other (FIG. 1, panel B). Hazard plots were not different between animals administered G. oxydans and animals administered A. aceti (FIG. 1, panel B).

Using the OASIS 2 software, restricted mean life span (RMLS) of animals administered either G. oxydans, A. aceti, or G. hansenii were calculated. The RMLS of animals administered E. coli OP50, 14.86±0.27 (RMLS±s.e, n=104) days, were significantly different compared to the RMLS of animals administered G. oxydans, 20.52±0.39 (RMLS±s.e, n=117) days, with two-tailed p value<0.0001. Similarly, the RMLS of animals administered E. coli OP50, 14.86±0.27 (RMLS±s.e, n=104) days, were significantly different compared to the RMLS of animals administered A. aceti, 19.17±0.41 (RMLS±s.e, n=124), with two-tailed p value<0.0001. Similarly, the RMLS of animals administered E. coli OP50, 14.86±0.27 (RMLS±s.e, n=104) days, were significantly different compared to the RMLS of animals administered G. hansenii, 22.95±0.39 (RMLS±s.e, n=103) days, with two-tailed p value<0.0001 (FIG. 1, panel C). A smaller but statistically significant difference (two-tailed p value=0.0181) were observed when the RMLS of animals administered G. oxydans compared to the RMLS of animals administered A. aceti. However, statistically significant differences (two-tailed p value<0.0001) were observed when RMLS of animals administered G. oxydans or A. aceti were compared to RMLS of animals administered G. hansenii. Comparing the survival curves of animals administered E. coli OP50 and either G. oxydans, A. aceti, or G. hansenii revealed significant differences in the survival rates (FIG. 1, panel D). Smaller but statistically different hazard rates were observed between animals administered either G. oxydans or A. aceti compared to animals administered G. hansenii (FIG. 1, panel D). However, the survival rates were not significantly different between animals administered G. oxydans compared to animals administered A. aceti (FIG. 1, panel D). From these observations, in the context of a lifespan assay, administration of G. hansenii significantly improved the lifespan of animals compared to E. coli OP50, G. oxydans or A. aceti.

### Example 2: Administration of Acetobacteraceae Improved Muscle Function/Activity

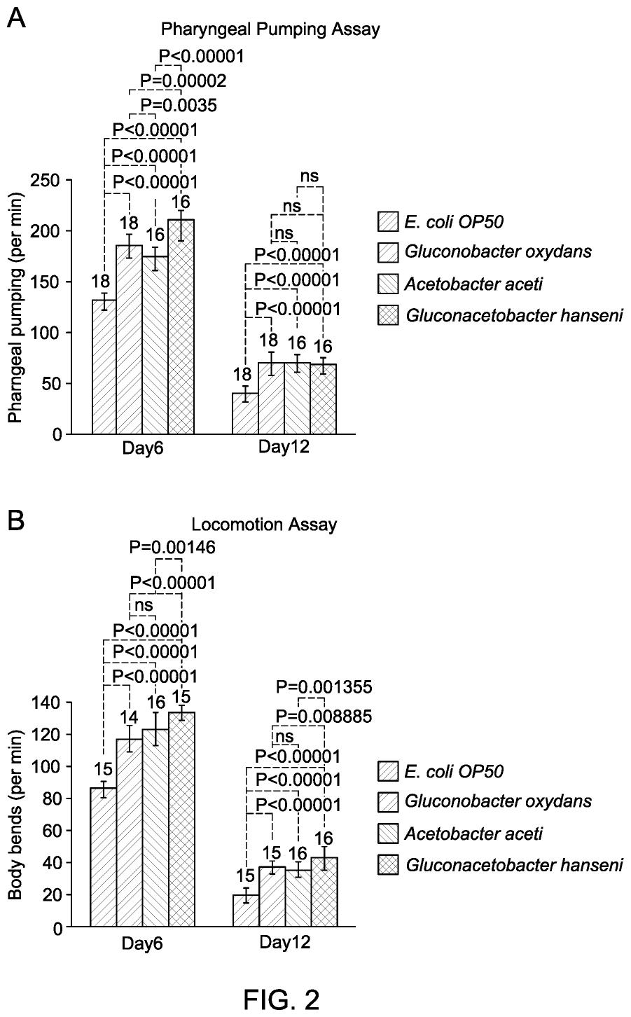

Since muscle function/activity decreases as animals grow old, pharyngeal pumping activity and locomotion rate in animals administered E. coli OP50, G. oxydans, A. aceti, or G. hansenii was monitored. Pharyngeal pumping activity was significantly decreased in 10-day old animals administered E. coli OP50 compared to 5-day old animals administered E. coli OP50 (FIG. 2, panel A). However, compared to animals administered E. coli OP50 animals, animals administered G. oxydans, A. aceti, or G. hansenii exhibited an improved pharyngeal pumping function whether it was in 5-day old animals or 10-day old animals (FIG. 2, panel A). This result suggests that the muscle function was better preserved in old animals administered G. oxydans, A. aceti, or G. hansenii compared to administered E. coli OP50. Pharyngeal pumping rates of 5-day old animals administered either G. oxydans, A. aceti, or G. hansenii were significantly higher than the rates in animals administered E. coli OP50 suggesting that the animals did not starve, and therefore, caloric restriction effect can be ruled out.

To analyze the efficacy of G. oxydans, A. aceti, or G. hansenii in delaying aging, the rate of locomotion during the time course of aging (6^{th} and 12^{th} day post adulthood) was measured. The locomotion rate was significantly decreased in 12-day old animals administered E. coli OP50 animals compared to 6-day old adults (FIG. 2, panel B; p-value<0.00001). However, in animals administered G. oxydans, A. aceti, or G. hansenii, the locomotion rate of 12-day old animals was significantly higher than in animals of same age administered E. coli OP50 (p-value<0.00001). Interestingly, the locomotion rate of 6-day old animals administered G. oxydans, A. aceti, or G. hansenii was significantly higher than in animals of same age administered E. coli OP50 (FIG. 2, panel B; p-value<0.00001). These results suggested that the neuromuscular function required for locomotion was better preserved in old animals administered G. hansenii.

### Example 3: Administration of Acetobacteraceae did not Impact Reproduction

Many long-lived C. elegans mutants exhibit a reduced reproductive capacity (Larsen et al., 1995)(Hughes et al., 2007). Therefore, the impact of G. hansenii on reproduction was tested. Fertility of animals administered G. hansenii was slightly lower than that of the animals administered E. coli OP50. The total number of progenies produced also decreased significantly in animals administered G. hansenii. Animals administered E. coli OP50 produced 180±31 (mean±s.d. of 15 animals) progeny, whereas 151±32 (mean±s.d. of 15 animals) progeny were produced by animals administered G. hansenii (two-tailed p value=0.0188). Measuring the time-course distribution of progeny production did not reveal any apparent differences in the rate of progeny production.

### Example 4: Administration of Acetobacteraceae improved stress resistance

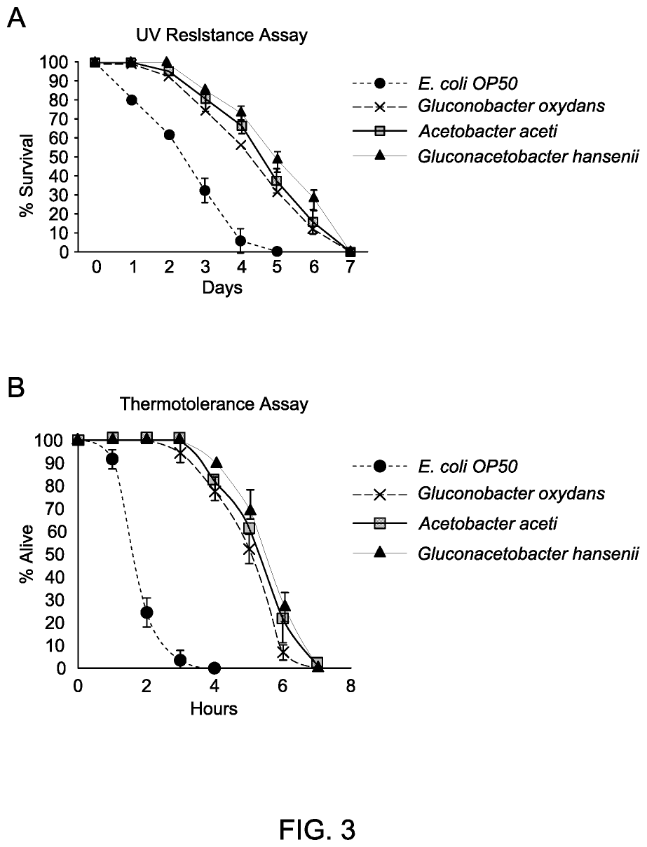

Since lifespan extension in C. elegans has been linked to stress resistance, the effect of G. oxydans, A. aceti, or G. hansenii administration on resistance to UV and thermotolerance was determined. Resistance to UV irradiation was significantly increased in animals administered either G. oxydans, A. aceti, or G. hansenii compared to animals administered either with E. coli OP50 (FIG. 3, panel A). Animals administered either with E. coli OP50, G. oxydans, A. aceti, or G. hansenii were exposed to UV irradiation (254 nm) at a dose of 1,000 J/m². The number of dead and viable animals were scored every day until all animals died. Animals administered either G. oxydans, A. aceti, or G. hansenii survived longer than the animals administered E. coli OP50 (FIG. 3, panel A). Mean survival times of wildtype animals administered either G. oxydans, A. aceti, or G. hansenii were 4.69±0.15 (RMLS±s.e, n=97, p<0.0001) days, 4.99±0.14 (RMLS±s.e, n=98, p<0.0001) days, and 5.4±0.14 (RMLS±s.e, n=98, p<0.0001) days respectively compared to 2.79±0.12 (RMLS±s.e, n=99) days in animals administered E. coli OP50.

To assess thermal shock stress resistance, the animals administered either with E. coli OP50, G. oxydans, A. aceti, or G. hansenii were transferred from 20° C. to 37° C. The viable and the dead nematodes were scored every hour until all animals died. Animals administered G. oxydans, A. aceti, or G. hansenii, extended the mean survival time of the animals after they were shifted to an elevated temperature significantly compared to the animals administered E. coli OP50. While >60% of wildtype animals administered E. coli OP50 died within 2 hours of shifting to 37° C., 100% of wildtype animals administered either G. oxydans, A. aceti, or G. hansenii were alive even after 4 hours of shifting to 37° C. (FIG. 3, panel B). Further, while 100% of wildtype animals administered E. coli OP50 died within 3 hours of shifting to 37° C., 100% of wildtype animals administered either G. oxydans, A. aceti, or G. hansenii died after 6 hours of shifting to 37° C. Thus, this data suggested that administration of G. oxydans, A. aceti, or G. hansenii conferred thermal stress resistance.

### Example 5: Administration of Acetobacteraceae Decreased Fat Deposition

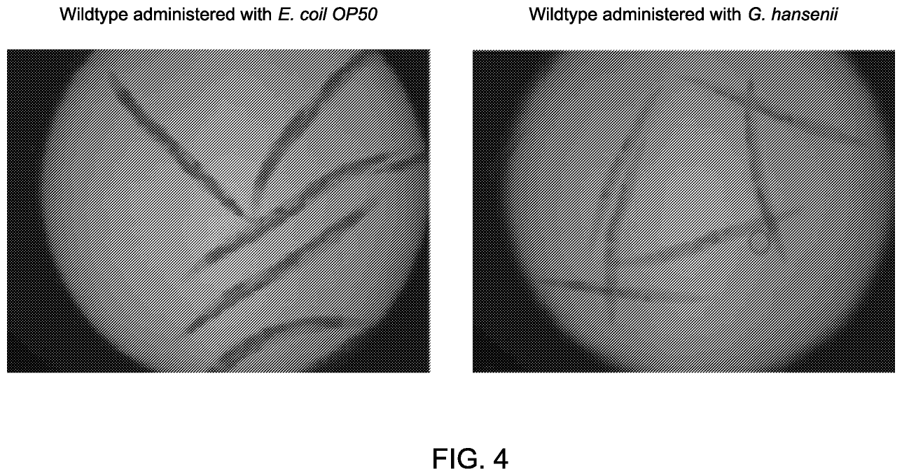

Aging can be associated with dysregulation of lipid metabolism in some animals. Therefore, effects of administration of G. hansenii on lipid levels with aging were tested. While large amounts of intestinal fat were observed with Oil red O staining in E. coli OP50-administered animals, this accumulation was not observed in G. hansenii-administered animals (FIG. 4).

### Example 6: prx-5, tcer-1 and aak-2 were Involved in G. hansenii-Induced Lifespan Extension

Because administration of G. hansenii had significant effect on various aspects of aging compared to G. oxydans or A. aceti, G. hansenii was focused on for further studies. Several conserved pathways including the insulin/IGF-1 like signaling (IIS) pathway (Tissenbaum and Ruvkun, 1998)(Kenyon, 2011), the target of rapamycin (TOR) (Robida-Stubbs et al., 2012)(Johnson et al., 2013), Nrf2/Antioxidant stress response pathway (Blackwell et al., 2015), TGF beta signaling (Kaplan et al., 2015)(Luo et al., 2010), Sirtuins (Dang, 2014)(Guarente, 2007)(Longo and Kennedy, 2006), autophagy(Gelino et al., 2016)(Hansen et al., 2008)(Chang et al., 2017), and the AMP-activated protein kinase (AMPK) pathways (Burkewitz et al., 2014)(Curtis et al., 2006)(Onken and Driscoll, 2010) have been reported to be involved in determining the lifespan in C. elegans. To identify the genetic pathways through which G. hansenii to improve lifespan, longevity assays on prx-5, tcer-1, aak-2 and daf-16 mutants were performed.

prx-5 encodes the ortholog of human PEXS, which is required for the peroxisomal import of cytosolic proteins containing peroxisomal targeting sequences (Wang et al., 2013). Peroxisome is an important organelle which plays an important role in several metabolic pathways including lipid metabolism. Age-dependent decline in peroxisomal protein import was observed previously (Narayan et al., 2016) and studies in yeast showed that reduction in peroxisomal import decreased the chronological lifespan (Lefevre et al., 2013). tcer-1 encodes a putative transcription elongation factor that regulates aging in C. elegans (Amrit et al., 2016)(Ghazi et al., 2009)(McCormick et al., 2012). aak-2 encodes an AMP-activated protein kinase that regulates lifespan in C. elegans (Curtis et al., 2006)(Moreno-Arriola et al., 2016)(Lee et al., 2008)(Apfeld et al., 2004). daf-16 encodes a FOXO-family transcription factor that functions downstream of insulin signaling to regulate lifespan in many animals including C. elegans (Kimura et al., 1997)(Murphy et al., 2003)(Lee et al., 2001).

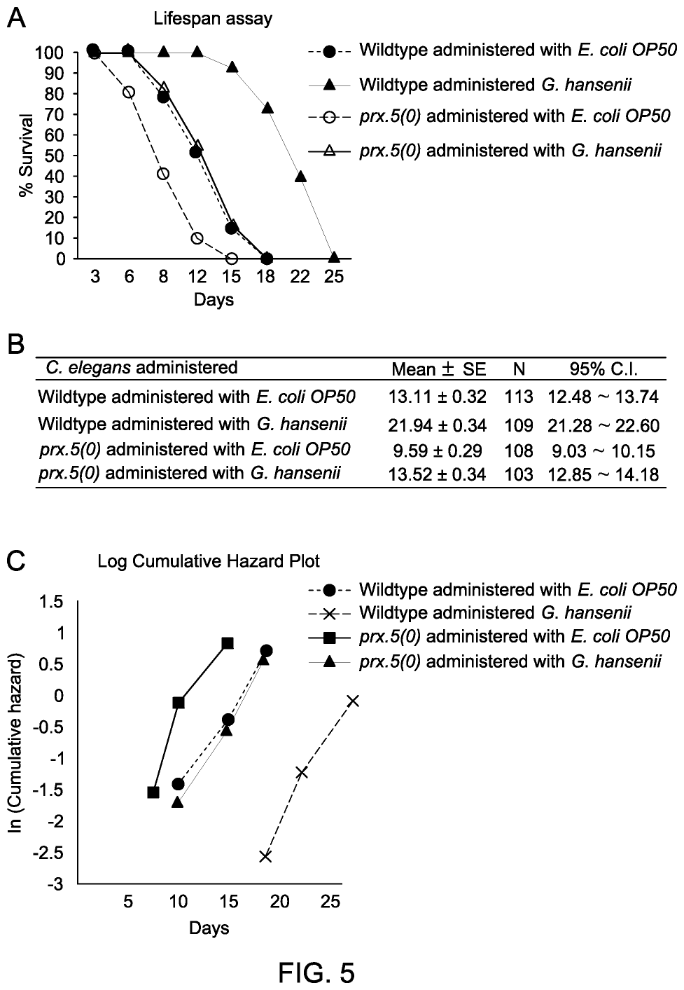

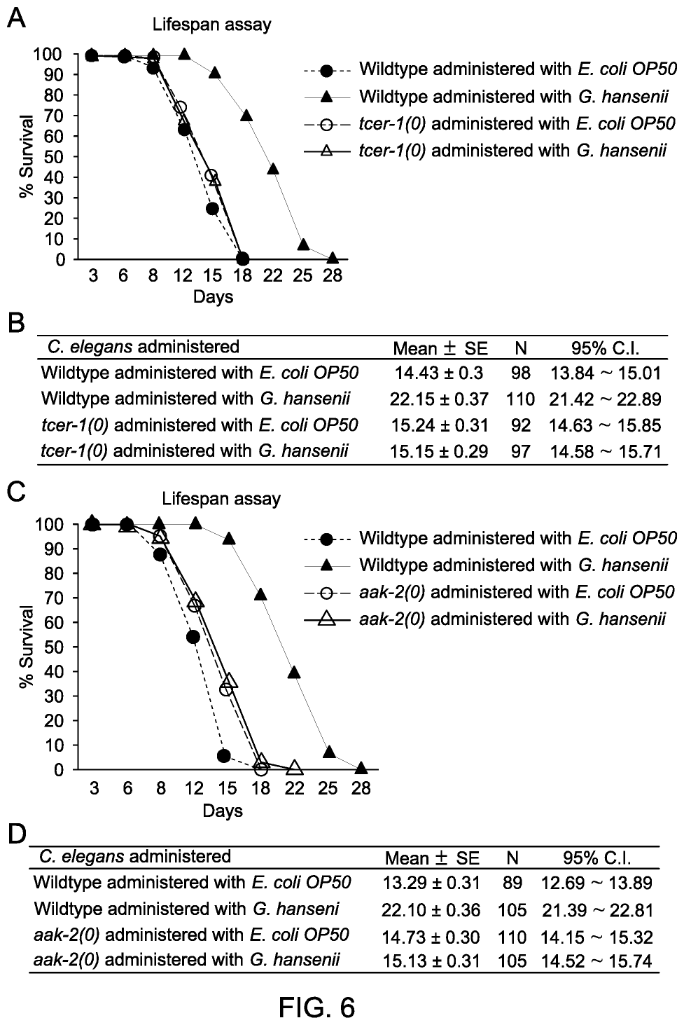

Based on data obtained, prx-5, tcer-1, and aak-2 were required for the G. hansenii-induced lifespan extension, but daf-16 was not required for the longevity phenotype. For the lifespan assays, a prx-5(ku517) strain in which PRX-5 is produced as truncated product (i.e., missing the last 26 amino acids of the protein (Wang et al., 2013)) was used. This strain is referred to herein as prx-5(0). Comparing the survival curves revealed that the RMLS of G. hansenii-administered wildtype animals was significantly higher than the RMLS of prx-5(0) animals administered G. hansenii [21.94±0.34(n=109) days vs 14.86±0.27(n=104) days, p<0.0001] (FIG. 5, panels A-B). The RMLS of prx-5(0) animals administered G. hansenii were more similar to that of wildtype animals administered E. coli OP50 [13.52±0.34(n=103) days vs 13.11±0.32(n=113) days, p=0.3805] (FIG. 5, panels A-B), which suggested that prx-5 was necessary for the G. hansenii-induced lifespan extension. Further, prx-5 appeared necessary for normal lifespan because prx-5(0) animals administered E. coli OP50 had decreased RMLS compared to that of wildtype animals administered E. coli OP50 [9.59±0.29 (n=108) days vs 13.11±0.32(n=113) days, p<0.0001] (FIG. 5, panels A-B). prx-5(0) animals administered G. hansenii had increased RMLS compared to prx-5(0) animals administered E. coli OP50 [13.52±0.34(n=103) days vs 9.59±0.29 (n=108) days, p<0.0001] (FIG. 5, panels A-B). This result suggested that although the lifespan extension in animals administered G. hansenii was dependent on prx-5, G. hansenii also improved the lifespan of prx-5(0) mutants. Similar results were obtained by comparing the cumulative hazard rate of wildtype and prx-5(0) animals administered either E. coli OP50 or G. hansenii (FIG. 5, panel C). Lifespan assays on tcer-1(0) animals revealed that while administration of G. hansenii extended the RMLS of wildtype animals compared to that of wildtype animals administered E. coli OP50 [22.15±0.37(n=110) days vs 14.43±0.30(n=98) days, p<0.0001], G. hansenii-administration did not extend the RMLS of tcer-1(0) animals compared to that of tcer-1(0) animals administered E. coli OP50 [15.15±0.29(n=97) days vs 15.24±0.31(n=92) days, p<0.0001] (FIG. 6, panel A-B). RMLS of tcer-1(0) animals administered G. hansenii was not significantly different from the RMLS of wildtype animals administered E. coli OP50 [15.15±0.29(n=97) days vs 14.43±0.3(n=98) days p=0.0861] or RMLS of of tcer-1(0) animals administered E. coli OP50 [15.15±0.29(n=97) days vs 15.24±0.31(n=92) days p=0.8322] (FIG. 6, panel A-B). This result suggested that tcer-1 was required for G. hansenii-induced lifespan extension. Lifespan assays on aak-2(0) animals revealed that while administration of G. hansenii extended the RMLS of wildtype animals compared to that of wildtype animals administered E. coli OP50 [22.10±0.36(n=105) days vs 13.29±0.31(n=89) days, p<0.0001], G. hansenii-administration did not extend the RMLS of aak-2(0) animals compared to that of aak-2(0) animals administered E. coli OP50 [15.13±0.31(n=105) days vs 14.73±0.30 (n=110) days, p<0.3548] (FIG. 6, panel C-D).

### Example 7: daf-16 was not Required for the G. hansenii-Induced Lifespan Extension

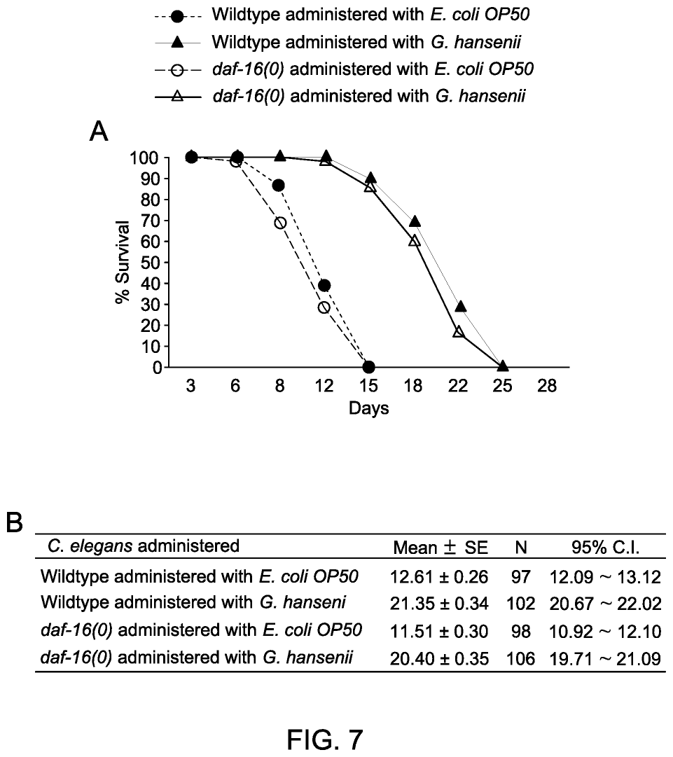

While prx-5, tcer-1 or aak-2 animals appeared to be necessary for G. hansenii-induced lifespan extension phenotype, results suggested that daf-16 was not required for the longevity phenotype. Lifespan assays revealed that the RIVILS of daf-16(0) animals administered G. hansenii was significantly increased compared to that of daf-16(0) animals administered E. coli OP50 [22.61±0.26(n=97) days vs 11.51±0.30(n=98) days, p<0.0001] (FIG. 7, panel A-B).

### Example 8: hsf-1 was Involved in G. hansenii-Induced Thermotolerance Phenotype

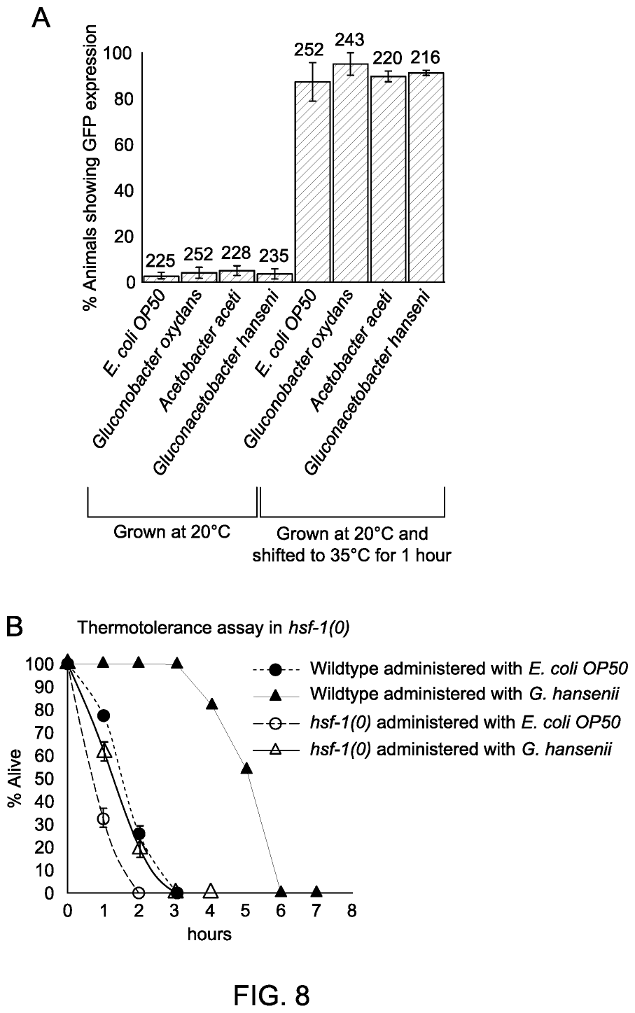

Thermotolerance in C. elegans has been associated with expression of heat shock proteins under the control of heat shock factor-1 (HSF-1) transcription factor (Haj du-Cronin et al., 2004)(Link et al., 1999); therefore, G. oxydans, A. aceti, and G. hansenii-administration were examined to determine if such administration would induce hsp-16.2::gfp expression. hsp-16.2 is a heat shock protein that is induced in heat stress in a heat shock factor-1 (HSF-1) transcription factor. While 2.5±1.1% (n=225) animals administered E. coli OP50 and grown at 20° C. showed hsp-16.2::gfp GFP induction, 86.6±8.3% (n=252) of animals administered E. coli OP50 and shifted to 35° C. for 1 hour had hsp-16.2::gfp expression. Animals administered either G. oxydans, A. aceti, or G. hansenii and grown at 20° C. did not show induction of hsp-16.2::gfp expression suggesting that induction of heat shock protein expression is not required for the thermotolerance phenotype. When the animals administered either G. oxydans, A. aceti, or G. hansenii were shifted to 35° C. for 1 hour, hsp-16.2::gfp expression was observed in 94.1±4.3% (n=243), 88.9±2.4% (n=220), 90.2±1.3% (n=216) of animals respectively (FIG. 8). This result suggested that administration with either G. oxydans, A. aceti, or G. hansenii did not affect the induction of heat shock response genes.

Although, induction of heat response genes in animals administered G. hansenii and grown at 20° C. was not observed, the thermotolerance phenotype was found to be dependent on HSF-1. While 100% of wildtype animals administered G. hansenii were alive even after 3 hours shifting to 37° C., 100% of hsf-1(0) animals administered G. hansenii were dead (FIG. 8, panel B). Further, while 25.6±4.7% (n=300) of wildtype animals administered E. coli OP50 were alive after 2 hours of shifting to 37° C., 100±0% (n=300) of hsf-1(0) animals administered E. coli OP50 were dead within 2 hours of shifting to 37° C., which suggested HSF-1 was required for thermotolerance (FIG. 8, panel B). Compared to the hsf-1(0) animals administered E. coli OP50 and shifted to 37° C., hsf-1(0) animals administered G. hansenii and shifted to 37° C. survived better (FIG. 8, panel B), which suggested that there might be HSF-1 independent pathways as well.

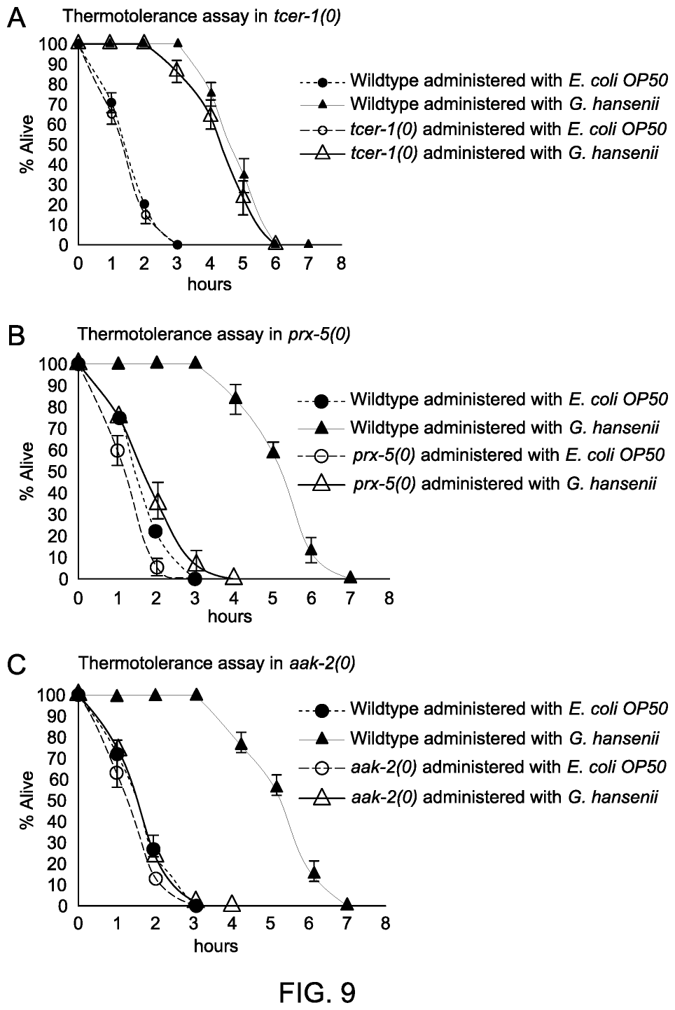

The results suggested that even though administration of G. hansenii did not induce hsp-16.2::gfp, the thermo-tolerance phenotype was dependent on HSF-1, as well as HSF-1 independent pathways. To test whether the thermotolerance phenotype of animals administered G. hansenii is dependent on PRX-5, TCER-1 or AAK-2, thermotolerance assays in tcer-1(0), prx-5(0) or aak-2(0) mutants were conducted. The survival curves of tcer-1(0) administered G. hansenii were similar to that of wildtype animals administered G. hansenii, which suggested that TCER-1 was not required for the thermotolerance phenotype (FIG. 9, panel A). prx-5(0) animals were found to be hypersensitive to heat stress compared to the wildtype; while 22±1% (n=300) of wildtype animals administered E. coli OP50 were alive after 2 hours of shifting to 37° C., 100±0% (n=300) of prx-5(0) animals administered E. coli OP50 were dead within 2 hours of shifting to 37° C. suggesting as PRX-5 is required for thermotolerance (FIG. 9, panel B). Further, while 100% of wildtype animals administered G. hansenii were alive even after 3 hours of shifting to 37° C., 100% of prx-5(0) animals administered G. hansenii were dead (FIG. 8, panel B), which suggested that PRX-5 was required for thermotolerance phenotype of animals administered G. hansenii. AAK-2 was found to be required for the thermotolerance phenotype of animals administered G. hansenii. Compared to the survival rate of wildtype animals administered G. hansenii, the survival rates of aak-2(0) animals administered G. hansenii was significantly reduced (FIG. 9, panel C). The survival curves of aak-2(0) animals administered E. coli OP50 was similar to that of wildtype animals administered E. coli OP50 suggesting that AAK-2 is not required for normal thermotolerance (FIG. 9, panel C).

### Other Embodiments

It is to be appreciated by those skilled in the art that various alterations, modifications, and improvements to the present disclosure will readily occur to those skilled in the art. Such alterations, modifications, and improvements are intended to be part of the present disclosure, and are intended to be within the spirit and scope of the invention. Accordingly, the foregoing description and drawing are by way of example only and any invention described in the present disclosure if further described in detail by the claims that follow.

Those skilled in the art will appreciate typical standards of deviation or error attributable to values obtained in assays or other processes as described herein. The publications, websites and other reference materials referenced herein to describe the background of the invention and to provide additional detail regarding its practice are hereby incorporated by reference in their entireties.

It is to be understood that while embodiments of the invention have been described in conjunction with the detailed description thereof, the foregoing description is intended to illustrate and not limit the scope of the invention, which is defined by the scope of the appended claims. Other aspects, advantages, and modifications are within the scope of the following claims.

### REFERENCES

All publications, patent applications, patents, and other references mentioned herein are incorporated by reference in their entirety.

Amrit, F. R. G., Steenkiste, E. M., Ratnappan, R., Chen, S.-W., McClendon, T. B., Kostka, D., Yanowitz, J., Olsen, C. P., and Ghazi, A. (2016). DAF-16 and TCER-1 Facilitate Adaptation to Germline Loss by Restoring Lipid Homeostasis and Repressing Reproductive Physiology in C. elegans. PLoS Genet. 12, e1005788.

An, R., Wilms, E., Masclee, A.A.M., Smidt, H., Zoetendal, E. G., and Jonkers, D. (2018). Age-dependent changes in GI physiology and microbiota: time to reconsider? Gut 67, 2213-2222.

Antebi, A. (2007). Genetics of Aging in Caenorhabditis elegans. PLOS Genet. 3, e129.

Apfeld, J., O'Connor, G., McDonagh, T., DiStefano, P. S., and Curtis, R. (2004). The AMP-activated protein kinase AAK-2 links energy levels and insulin-like signals to lifespan in C. elegans. Genes Dev. 18, 3004-3009.

Arey, R. N., and Murphy, C. T. (2017). Conserved regulators of cognitive aging: From worms to humans. Behay. Brain Res. 322, 299-310.

Bansal, A., Zhu, L. J., Yen, K., and Tissenbaum, H. A. (2015). Uncoupling lifespan and healthspan in Caenorhabditis elegans longevity mutants. Proc. Natl. Acad. Sci. 112, E277-E286.

Beltrán-Sánchez, H., Soneji, S., and Crimmins, E. M. (2015). Past, Present, and Future of Healthy Life Expectancy. Cold Spring Harb. Perspect. Med. 5.

Bischoff, S. C. (2016). Microbiota and aging. Curr. Opin. Clin. Nutr. Metab. Care 19, 26-30.

Bitto, A., Wang, A. M., Bennett, C. F., and Kaeberlein, M. (2015). Biochemical Genetic Pathways that Modulate Aging in Multiple Species. Cold Spring Harb. Perspect. Med. 5.

Blackwell, T. K., Steinbaugh, M. J., Hourihan, J. M., Ewald, C. Y., and Isik, M. (2015). SKN-1/Nrf, stress responses, and aging in Caenorhabditis elegans. Free Radic. Biol. Med. 88, 290-301.

Brooks, K. K., Liang, B., and Watts, J. L. (2009). The influence of bacterial diet on fat storage in C. elegans. PloS One 4, e7545.

Burkewitz, K., Zhang, Y., and Mair, W. B. (2014). AMPK at the Nexus of Energetics and Aging. Cell Metab. 20, 10-25.

Cabreiro, F., Au, C., Leung, K.-Y., Vergara-Irigaray, N., Cochemé, H. M., Noori, T., Weinkove, D., Schuster, E., Greene, N. D. E., and Gems, D. (2013). Metformin retards aging in C. elegans by altering microbial folate and methionine metabolism. Cell 153, 228-239.

Chang, J. T., Kumsta, C., Hellman, A. B., Adams, L. M., and Hansen, M. (2017). Spatiotemporal regulation of autophagy during Caenorhabditis elegans aging. ELife 6.

Choi, J., Hur, T.-Y., and Hong, Y. (2018). Influence of Altered Gut Microbiota Composition on Aging and Aging-Related Diseases. J. Lifestyle Med. 8, 1-7.

Claesson, M. J., Jeffery, L B., Conde, S., Power, S. E., O'Connor, E. M., Cusack, S., Harris, H. M. B., Coakley, M., Lakshminarayanan, B., O'Sullivan, O., et al. (2012). Gut microbiota composition correlates with diet and health in the elderly. Nature 488, 178-184.

Collins, J. J., Evason, K., and Kornfeld, K. (2006). Pharmacology of delayed aging and extended lifespan of Caenorhabditis elegans. Exp. Gerontol. 41, 1032-1039.

Curtis, R., O'Connor, G., and DiStefano, P. S. (2006). Aging networks in Caenorhabditis elegans: AMP-activated protein kinase (aak-2) links multiple aging and metabolism pathways. Aging Cell 5,119-126.

Dang, W. (2014). The controversial world of sirtuins. Drug Discov. Today Technol. 12, e9—e17.

Denzel, M. S., Lapierre, L. R., and Mack, H. I. D. (2019). Emerging topics in C. elegans aging research: Transcriptional regulation, stress response and epigenetics. Mech. Ageing Dev. 177, 4-21.

Dirksen, P., Marsh, S. A., Braker, I., Heitland, N., Wagner, S., Nakad, R., Mader, S., Petersen, C., Kowallik, V., Rosenstiel, P., et al. (2016). The native microbiome of the nematode Caenorhabditis elegans: gateway to a new host-microbiome model. BMC Biol. 14, 38.

Donato, V., Ayala, F. R., Cogliati, S., Bauman, C., Costa, J. G., Lenini, C., and Grau, R. (2017). Bacillus subtilis biofilm extends Caenorhabditis elegans longevity through downregulation of the insulin-like signalling pathway. Nat. Commun. 8, 14332.

Ezcurra, M. (2018). Dissecting cause and effect in host-microbiome interactions using the combined worm-bug model system. Biogerontology 19, 567-578.

Fasseas, M. K., Fasseas, C., Mountzouris, K. C., and Syntichaki, P. (2013). Effects of Lactobacillus salivarius, Lactobacillus reuteri, and Pediococcus acidilactici on the nematode Caenorhabditis elegans include possible antitumor activity. Appl. Microbiol. Biotechnol. 97, 2109-2118.

Félix, M.-A., and Braendle, C. (2010). The natural history of Caenorhabditis elegans. Curr. Biol. CB 20, R965-969.

Finch, C. E., and Ruvkun, G. (2001). The Genetics of Aging. Annu. Rev. Genomics Hum. Genet. 2, 435-462.

Franceschi, C., Garagnani, P., Morsiani, C., Conte, M., Santoro, A., Grignolio, A., Monti, D., Capri, M., and Salvioli, S. (2018). The Continuum of Aging and Age-Related Diseases: Common Mechanisms but Different Rates. Front. Med. 5.

Frasca, D., Blomberg, B. B., and Paganelli, R. (2017). Aging, Obesity, and Inflammatory Age-Related Diseases. Front. Immunol. 8.

Gelino, S., Chang, J. T., Kumsta, C., She, X., Davis, A., Nguyen, C., Panowski, S., and Hansen, M. (2016). Intestinal Autophagy Improves Healthspan and Longevity in C. elegans during Dietary Restriction. PLOS Genet. 12, e1006135.

Gerber, G. K. (2014). The dynamic microbiome. FEB S Lett. 588, 4131-4139.

Ghazi, A., Henis-Korenblit, S., and Kenyon, C. (2009). A transcription elongation factor that links signals from the reproductive system to lifespan extension in Caenorhabditis elegans. PLoS Genet. 5, e1000639.

Grompone, G., Martorell, P., Llopis, S., González, N., Genovés, S., Mulet, A. P., Fernandez-Calero, T., Tiscornia, I., Bollati-Fogolín, M., Chambaud, I., et al. (2012). Anti-inflammatory Lactobacillus rhamnosus CNCM 1-3690 strain protects against oxidative stress and increases lifespan in Caenorhabditis elegans. PloS One 7, e52493.

Guarente, L. (2007). Sirtuins in aging and disease. Cold Spring Harb. Symp. Quant. Biol. 72, 483-488.

Hajdu-Cronin, Y. M., Chen, W. J., and Sternberg, P. W. (2004). The L-Type Cyclin CYL-1 and the Heat-Shock-Factor HSF-1 Are Required for Heat-Shock-Induced Protein Expression in Caenorhabditis elegans. Genetics 168, 1937-1949.

Han, S. K., Lee, D., Lee, H., Kim, D., Son, H. G., Yang, J.-S., Lee, S.-J. V., and Kim, S. (2016). OASIS 2: online application for survival analysis 2 with features for the analysis of maximal lifespan and healthspan in aging research. Oncotarget 7,56147-56152.

Hansen, M., Chandra, A., Mitic, L. L., Onken, B., Driscoll, M., and Kenyon, C. (2008). A Role for Autophagy in the Extension of Lifespan by Dietary Restriction in C. elegans. PLOS Genet. 4, e24.

Heintz, C., and Mair, W. (2014). You Are What You Host: Microbiome Modulation of the Aging Process. Cell 156, 408-411.

Hughes, S. E., Evason, K., Xiong, C., and Kornfeld, K. (2007). Genetic and pharmacological factors that influence reproductive aging in nematodes. PLoS Genet. 3, e25.

Johnson, T. E. (2003). Advantages and disadvantages of Caenorhabditis elegans for aging research. Exp. Gerontol. 38, 1329-1332.

Johnson, S. C., Rabinovitch, P. S., and Kaeberlein, M. (2013). mTOR is a key modulator of ageing and age-related disease. Nature 493, 338-345.

Jonassen, T., Larsen, P. L., and Clarke, C. F. (2001). A dietary source of coenzyme Q is essential for growth of long-lived Caenorhabditis elegans clk-1 mutants. Proc. Natl. Acad. Sci. 98, 421-426.

Kapahi, P., Kaeberlein, M., and Hansen, M. (2017). Dietary restriction and lifespan: Lessons from invertebrate models. Ageing Res. Rev. 39, 3-14.

Kaplan, R. E. W., Chen, Y., Moore, B. T., Jordan, J. M., Maxwell, C. S., Schindler, A. J., and Baugh, L. R. (2015). db1-1/TGF-β and daf-12/NHR Signaling Mediate Cell-Nonautonomous Effects of daf-16/FOXO on Starvation-Induced Developmental Arrest. PLOS Genet. 11, e1005731.

Kenyon, C. (2005). The Plasticity of Aging: Insights from Long-Lived Mutants. Cell 120, 449-460.

Kenyon, C. (2011). The first long-lived mutants: discovery of the insulin/IGF-1 pathway for ageing. Philos. Trans. R. Soc. Lond. B. Biol. Sci. 366, 9-16.

Kim, S., and Jazwinski, S. M. (2018). The Gut Microbiota and Healthy Aging: A Mini-Review. Gerontology 64, 513-520.

Kimura, K. D., Tissenbaum, H. A., Liu, Y., and Ruvkun, G. (1997). daf-2, an insulin receptor-like gene that regulates longevity and diapause in Caenorhabditis elegans. Science 277, 942-946.

Knickman, J. R., and Snell, E. K. (2002). The 2030 problem: caring for aging baby boomers. Health Serv. Res. 37,849-884.

Komura, T., Ikeda, T., Yasui, C., Saeki, S., and Nishikawa, Y. (2013). Mechanism underlying prolongevity induced by bifidobacteria in Caenorhabditis elegans. Biogerontology 14, 73-87.

Larsen, P. L., Albert, P. S., and Riddle, D. L. (1995). Genes that regulate both development and longevity in Caenorhabditis elegans. Genetics 139, 1567-1583.

Lee, H., Cho, J. S., Lambacher, N., Lee, J., Lee, S.-J., Lee, T. H., Gartner, A., and Koo, H.-S. (2008). The Caenorhabditis elegans AMP-activated Protein Kinase AAK-2 Is Phosphorylated by LKB1 and Is Required for Resistance to Oxidative Stress and for Normal Motility and Foraging Behavior. J. Biol. Chem. 283, 14988-14993.

Lee, R. Y. N., Hench, J., and Ruvkun, G. (2001). Regulation of C. elegans DAF-16 and its human ortholog FKHRL1 by the daf-2 insulin-like signaling pathway. Curr. Biol. 11, 1950-1957.

Lefevre, S. D., van Roermund, C. W., Wanders, R. J. A., Veenhuis, M., and van der Klei, I. J. (2013). The significance of peroxisome function in chronological aging of Saccharomyces cerevisiae. Aging Cell 12, 784-793.

Link, C. D., Cypser, J. R., Johnson, C. J., and Johnson, T. E. (1999). Direct observation of stress response in Caenorhabditis elegans using a reporter transgene. Cell Stress Chaperones 4, 235-242.

Longo, V. D., and Kennedy, B. K. (2006). Sirtuins in Aging and Age-Related Disease. Cell 126, 257-268.

Lunenfeld, B., and Stratton, P. (2013). The clinical consequences of an ageing world and preventive strategies. Best Pract. Res. Clin. Obstet. Gynaecol. 27, 643-659.

Luo, S., Kleemann, G. A., Ashraf, J. M., Shaw, W. M., and Murphy, C. T. (2010). TGF-β and insulin signaling regulate reproductive aging via oocyte and germline quality maintenance. Cell 143, 299-312.

MacNeil, L. T., and Walhout, A. J. (2013). Food, pathogen, signal. Worm 2.

Maffei, V. J., Kim, S., Blanchard, E., Luo, M., Jazwinski, S. M., Taylor, C. M., and Welsh, D. A. (2017). Biological Aging and the Human Gut Microbiota. J. Gerontol. A. Biol. Sci. Med. Sci. 72, 1474-1482.

Maier, W., Adilov, B., Regenass, M., and Alcedo, J. (2010). A Neuromedin U Receptor Acts with the Sensory System to Modulate Food Type-Dependent Effects on C. elegans Lifespan. PLoS Biol. 8.

Martorell, P., Llopis, S., González, N., Chenoll, E., López-Carreras, N., Aleixandre, A., Chen, Y., Karoly, E. D., Ramón, D., and Genovés, S. (2016). Probiotic Strain Bifidobacterium animalis subsp. lactis CECT 8145 Reduces Fat Content and Modulates Lipid Metabolism and Antioxidant Response in Caenorhabditis elegan. J. Agric. Food Chem. 64, 3462-3472.

Maynard, C., Cummins, I., Green, J., and Weinkove, D. (2018). A bacterial route for folic acid supplementation. BMC Biol. 16, 67.

McCormick, M., Chen, K., Ramaswamy, P., and Kenyon, C. (2012). New genes that extend Caenorhabditis elegans lifespan in response to reproductive signals. Aging Cell 11, 192-202.

Moreno-Arriola, E., Hafidi, M. E., Ortega-Cuellar, D., and Carvajal, K. (2016). AMP-Activated Protein Kinase Regulates Oxidative Metabolism in Caenorhabditis elegans through the NHR-49 and MDT-15 Transcriptional Regulators. PLOS ONE 11, e0148089.

Murphy, C. T., McCarroll, S. A., Bargmann, C. I., Fraser, A., Kamath, R. S., Ahringer, J., Li, H., and Kenyon, C. (2003). Genes that act downstream of DAF-16 to influence the lifespan of Caenorhabditis elegans. Nature 424, 277-283.

Narayan, V., Ly, T., Pourkarimi, E., Murillo, A. B., Gartner, A., Lamond, A. I., and Kenyon, C. (2016). Deep Proteome Analysis Identifies Age-Related Processes in C. elegans. Cell Syst. 3,144-159.

Onken, B., and Driscoll, M. (2010). Metformin Induces a Dietary Restriction— Like State and the Oxidative Stress Response to Extend C. elegans Healthspan via AMPK, LKB1, and SKN-1. PLOS ONE 5, e8758.

Pan, H., and Finkel, T. (2017). Key proteins and pathways that regulate lifespan. J. Biol. Chem. 292, 6452-6460.

Pang, S., and Curran, S. P. (2014). Adaptive Capacity to Bacterial Diet Modulates Aging in C. elegans. Cell Metab. 19, 221-231.

Robida-Stubbs, S., Glover-Cutter, K., Lamming, D. W., Mizunuma, M., Narasimhan, S.D., Neumann-Haefelin, E., Sabatini, D. M., and Blackwell, T. K. (2012). TOR signaling and rapamycin influence longevity by regulating SKN-1/Nrf and DAF-16/FoxO. Cell Metab. 15, 713-724.

Samuel, B. S., Rowedder, H., Braendle, C., Felix, M.-A., and Ruvkun, G. (2016). Caenorhabditis elegans responses to bacteria from its natural habitats. Proc. Natl. Acad. Sci. 113, E3941—E3949.

Smith, E. D., Kaeberlein, T. L., Lydum, B. T., Sager, J., Welton, K. L., Kennedy, B. K., and Kaeberlein, M. (2008). Age- and calorie-independent life span extension from dietary restriction by bacterial deprivation in Caenorhabditis elegans. BMC Dev. Biol. 8, 49.

Soukas, A. A., Kane, E. A., Carr, C. E., Melo, J. A., and Ruvkun, G. (2009). Rictor/TORC2 regulates fat metabolism, feeding, growth, and life span in Caenorhabditis elegans. Genes Dev. 23, 496-511.

Srivastava, S. (2017). The Mitochondrial Basis of Aging and Age-Related Disorders. Genes 8.

Sugawara, T., and Sakamoto, K. (2018). Killed Bifidobacterium longum enhanced stress tolerance and prolonged life span of Caenorhabditis elegans via DAF-16. Br. J. Nutr. 120, 872-880.

Tilg, H., and Kaser, A. (2011). Gut microbiome, obesity, and metabolic dysfunction. J. Clin. Invest. 121, 2126-2132.

Tissenbaum, H. A., and Ruvkun, G. (1998). An insulin-like signaling pathway affects both longevity and reproduction in Caenorhabditis elegans. Genetics 148, 703-717.

Vaiserman, A. M., Koliada, A. K., and Marotta, F. (2017). Gut microbiota: A player in aging and a target for anti-aging intervention. Ageing Res. Rev. 35, 36-45.

Wang, R., Kniazeva, M., and Han, M. (2013). Peroxisome Protein Transportation Affects Metabolism of Branched-Chain Fatty Acids That Critically Impact Growth and Development of C. elegans. PLoS ONE 8.

Wilkinson, D. S., Taylor, R. C., and Dillin, A. (2012). Analysis of aging in Caenorhabditis elegans. Methods Cell Biol. 107, 353-381.

Zapata, H. J., and Quagliarello, V. J. (2015). The microbiota and microbiome in aging: potential implications in health and age-related diseases. J. Am. Geriatr. Soc. 63, 776-781.

Zhao, L., Zhao, Y., Liu, R., Zheng, X., Zhang, M., Guo, H., Zhang, H., and Ren, F. (2017). The Transcription Factor DAF-16 is Essential for Increased Longevity in C. elegans Exposed to Bifidobacterium longum BB68. Sci. Rep. 7, 7408.

Zhao, Y., Zhao, L., Zheng, X., Fu, T., Guo, H., and Ren, F. (2013). Lactobacillus salivarius strain FDB89 induced longevity in Caenorhabditis elegans by dietary restriction. J. Microbiol. Seoul Korea 51, 183-188.

What is claimed is:

1. A composition comprising:
(a) at least one bacterial strain or extracts or components thereof, wherein the at least one bacterial strain comprises Gluconobacter spp., Acetobacter spp., Gluconoacaetobacter spp., Acidomonas spp., Ameyamaea spp., Asaia spp., Granulibacter spp Kozakia spp Neoasaia spp., Neokomagataea spp., Saccharibacter spp., Swaminathania spp., Tanticharoenia spp., or a combination thereof; and
(b) an excipient.

2. The composition of claim 1, wherein the at least one bacterial strain comprises Gluconacetobacter hansenii, Gluconobacter oxydans, Acetobacter aceti, or a combination thereof.

3. The composition of claim 1 or 2, wherein the at least one bacterial strain comprises Gluconacetobacter hansenii.

4. The composition of any one of claims 1-3, wherein the at least one bacterial strain or extracts or components thereof is characterized in that, when administered to a C. elegans culture comprising C. elegans animals, an average life span of the C. elegans animals in the C. elegans culture is extended by at least 20%, as compared to that of C. elegans animals in a comparable C. elegans culture without administration of the at least one bacterial strain or extracts or components thereof.

5. The composition of any one of claims 1-4, wherein the at least one bacterial strain or extracts or components thereof is characterized in that, when administered to a C. elegans culture comprising C. elegans animals, average pharyngeal pumping activity of the C. elegans animals in the C. elegans culture is increased by at least 20%, as compared to that of C. elegans animals in a comparable C. elegans culture without administration of the at least one bacterial strain or extracts or components thereof.

6. The composition of any one of claims 1-5, wherein the at least one bacterial strain or extracts or components thereof is characterized in that, when administered to a C. elegans culture comprising C. elegans animals, average locomotion rate of the C. elegans animals in the C. elegans culture is increased by at least 20%, as compared to that of C. elegans animals in a comparable C. elegans culture without administration of the at least one bacterial strain or extracts or components thereof.

7. The composition of any one of claims 1-6, wherein the at least one bacterial strain or extracts or components thereof is characterized in that, when administered to a C. elegans culture comprising C. elegans animals, fertility of the C. elegans animals in the C. elegans culture is decreased by at least 20%, as compared to that of C. elegans animals in a comparable C. elegans culture without administration of the at least one bacterial strain or extracts or components thereof.

8. The composition of any one of claims 1-7, wherein the at least one bacterial strain or extracts or components thereof is characterized in that, when a C. elegans culture comprising C. elegans animals is exposed to Ultra Violet irradiation, average survival time of the C. elegans animals in the C. elegans culture to which the at least one bacterial strain or extracts or components thereof has been administered is increased by at least 20%, as compared to that of C. elegans animals in a comparable C. elegans culture without administration of the at least one bacterial strain or extracts or components thereof.

9. The composition of any one of claims 1-8, wherein the at least one bacterial strain or extracts or components thereof is characterized in that, when a C. elegans culture comprising C. elegans animals is exposed to an elevated temperature, average survival time of C. elegans animals in the C. elegans culture to which the at least one bacterial strain or extracts or components thereof has been administered is increased by at least 20%, as compared to that C. elegans animals in of a comparable C. elegans culture without administration of the at least one bacterial strain or extracts or components thereof.

10. The composition of claim 9, wherein the elevated temperature is at least 37° C.

11. The composition of any one of claims 1-10, wherein the at least one bacterial strain or extracts or components thereof is characterized in that, when administered to a C. elegans culture comprising C. elegans animals, average amount of intestinal fat observed in the C. elegan animals in the C. elegans culture is decreased by at least 20%, as compared to that of C. elegans animals in a comparable C. elegans culture without administration of the at least one bacterial strain or extracts or components thereof.

12. The composition of any one of claims 4-11, wherein the C. elegans animals are adult C. elegans animals.

13. The composition of any one of claims 4-12, wherein the C. elegans animals are at least 5 days old.

14. The composition of any one of claims 1-13, wherein the composition is formulated for oral administration.

15. The composition of any one of claims 1-14, wherein the composition is a food, a beverage, a feed composition, or a nutritional supplement.

16. The composition of any one of claims 1-15, wherein the composition is a liquid, syrup, tablet, troche, gummy, capsule, powder, gel, or film.

17. The composition of any one of claims 1-16, wherein the composition is a pharmaceutical composition.

18. The composition of any one of claims 1-17, wherein the composition is an enteric-coated formulation.

19. A method comprising administering to a subject a composition of any one of claims 1-18.

20. The method of claim 19, wherein the method is a method of extending lifespan of a subject.

21. The method of claim 20, wherein the life span of the subject is extended by at least 20%, as compared to that of a comparable subject without administration of the composition.

22. The method of claim 19, wherein the method is a method of reducing or delaying the onset of at least one age-associated symptom or condition in a subject.

23. The method of claim 22, wherein the at least one age-associated symptom or condition is reduced or delayed in the subject by at least 20%, as compared to that of a comparable subject without administration of the composition.

24. The method of claim 22 or 23, wherein the at least one age-associated symptom or condition is or comprises a decline in muscle and/or neuromuscular function of the subject.

25. The method of any one of claims 22-24, wherein the at least one age-associated symptom or condition is or comprises dysregulation of lipid metabolism.

26. The method of any one of claims 19-25, wherein the subject is at least 30 years old.

27. The method of any one of claims 19-26, wherein the subject is an elderly subject.

28. The method of any one of claims 19-27, wherein the subject is a mammal.

29. The method of any one of claims 19-28, wherein the subject is a human.

30. The method of any one claims 19-29, wherein administering comprises administering a sufficient amount of the microbe to colonize the subject's microbiome.

31. Use of at least one bacterial strain or extracts or components thereof for extending the life span of a subject, wherein at least one bacterial strain comprises Gluconobacter spp., Acetobacter spp., Gluconoacaetobacter spp., Acidomonas spp., Ameyamaea spp., Asaia spp., Granulibacter spp., Kozakia spp., Neoasaia spp., Neokomagataea spp Saccharibacter spp., Swaminathania spp., Tanticharoenia spp., or a combination thereof.

32. The use of claim 31, wherein the at least one bacterial strain comprises Gluconacetobacter hansenii, Gluconobacter oxydans, Acetobacter aceti, or a combination thereof.

33. The use of claim 31 or 32, wherein the at least one bacterial strain comprises Gluconacetobacter hansenii.

34. Use of at least one bacterial strain or extracts or components thereof reducing or delaying the onset of at least one age-associated symptom or condition in a subject, wherein the at least one bacterial strain comprises Gluconobacter spp., Acetobacter spp., Gluconoacaetobacter spp., Acidomonas spp., Ameyamaea spp., Asaia spp Granulibacter spp., Kozakia spp., Neoasaia spp., Neokomagataea spp., Saccharibacter spp., Swaminathania spp., Tanticharoenia spp., or a combination thereof.

35. The use of claim 34, wherein the at least one bacterial strain comprises Gluconacetobacter hansenii, Gluconobacter oxydans, Acetobacter aceti, or a combination thereof.

36. The use of claim 34 or 35, wherein the at least one bacterial strain comprises Gluconacetobacter hansenii.

37. The use of claim 34, wherein the at least one age-associated symptom or condition is reduced or delayed in the subject by at least 20%, as compared to that of a comparable subject without administration of the composition.

38. The use of any one of claims 34-37, wherein the at least one age-associated symptom or condition is or comprises a decline in muscle and/or neuromuscular function of the subject.

39. The use of any one of claims 34-38, wherein the at least one age-associated symptom or condition is or comprises dysregulation of lipid metabolism.

40. The use of any one of claims 34-39, wherein the subject is at least 30 years old.

41. The use of any one of claims 34-40, wherein the subject is an elderly subject.

42. The use of any one of claims 34-41, wherein the subject is a mammal.

43. The use of any one of claims 34-42, wherein the subject is a human.

44. A method of characterizing the ability of one or more microbial strains to modify the life span of a subject, an age-associated symptom, and/or an age-associated condition, comprising:
(a) adding a plurality of microbial strains of a mammalian microbiome to a plurality of C. elegans cultures, wherein a different microbial strain is added to each C. elegans culture, and wherein each culture comprises C. elegans animals of the same C. elegans strain, and
(b) determining whether each microbial strain of the plurality affects one or more parameters of the C. elegans animals of each culture, wherein the one or more parameters are associated with aging, an age-associated symptom, and/or an age-associated condition.

45. Use of C. elegans animals for characterizing the ability of one or more one or more microbial strains to modify the life span of a subject, an age-associated symptom, and/or an age-associated condition.

46. A method of making a composition according to any one of claims 1-18 comprising combining at least one bacterial strain or extracts or components thereof and the excipient.
